(19) United States
(12) Patent Application Publication
(10) Pub. No.: US 2026/0357339 A1
(43) Pub. Date: Nov. 12, 2026

(54) ANALYSIS OF A POLYMER

(71) Applicant: Oxford Nanopore Technologies PLC, Oxford (GB)

(72) Inventors: Graham James Hall, Oxford (GB); Miguel Ângelo Freitas Ribeiro Gaspar Reis, Oxford (GB); Steven Pool, Oxford (GB)

(73) Assignee: Oxford Nanopore Technologies PLC, Oxford (GB)

(21) Appl. No.: 19/122,602

(22) PCT Filed: Oct. 19, 2023
(86) PCT No.: PCT/GB2023/052708

§ 371 Date: Apr. 18, 2025

(30) Foreign Application Priority Data
Oct. 19, 2022 (GB) 2215442.1

(51) Int. Cl. C12Q 1/6874

(52) U.S. Cl. CPC C12Q 1/6874

(57) ABSTRACT

A nanopore device and a method of controlling a nanopore device for analysing a polymer, the nanopore device comprising at least one sensor element, the sensor element comprising a nanopore and a sensor, the method comprising: translocating a polymer through the nanopore; generating a series of measurements using the sensor as the polymer translocates through the nanopore; comparing the series of measurements to a reference data to determine a measurement of similarity; operating the nanopore device to eject the polymer from the nanopore if the measure of similarity is determined to be below a threshold value; and determining whether the polymer has been successfully ejected from the nanopore by analysing a further set of measurements taken by the sensor.

The present invention relates to the analysis of polymer analytes via the control of a nanopore device. More specifically, the present invention relates to the control of a nanopore device comprising a sensor element. The polymer may be, for example but without limitation, a polynucleotide in which the polymer units are nucleotides.

The use of nanopores to sense interactions with molecular entities, for example polynucleotides is a powerful technique that has been subject to much recent development. Nanopore devices have been developed that comprise an array of nanopore sensing elements, thereby increasing data collection by allowing plural nanopores to sense interactions in parallel, typically from the same sample.

Nanopore devices may typically employ an electrical signal across a nanopore channel to generate a measurement signal that is interpreted to sense and/or characterise molecular entities as they interact with the nanopore. Typically an electrical signal is applied as a potential difference or current across the array of sensor elements (also referred to as nanopore channels) that will provide a meaningful measurement signal to be interpreted. The measurement can include, for example, one of ionic current flow, electrical resistance, or voltage.

Such nanopore devices can provide long continuous reads of polymers, for example in the case of polynucleotides ranging from many hundreds to tens of thousands (and potentially more) nucleotides. The data gathered in this way comprises measurements, such as measurements of ion current, where each translocation of the sequence through the sensitive part of the nanopore results in a slight change in the measured property.

Whilst such nanopore devices can provide significant advantages, it remains desirable to increase the speed of analysis and the efficiency in terms of usage of the available sensor elements in the nanopore device.

According to a first aspect of the invention, there is provided a method of controlling a nanopore device for analysing a polymer, the nanopore device comprising at least one sensor element, the sensor element comprising a nanopore and a sensor electrode, the method comprising: translocating a polymer through the nanopore; determining a series of measurements from the sensor electrode as the polymer translocates through the nanopore; analysing the series of measurements against at least one reference sequence to determine a measurement of similarity; responding to the measure of similarity, operating the nanopore device to eject the polymer from the sensor element; and determining whether the sensor element is free from polymer by analysing measurements taken by the sensor electrode.

The polymer may comprise a series of polymer units to be identified by the nanopore device. It has been found that the desire to increase speed of analysis, especially in polymers with a higher number of polymer units (i.e. polymers of longer lengths), can lead to unforeseen inefficiencies in the nanopore device. One particular method that can be employed to increase the speed of analysis, particularly during analysis of polymers of longer lengths, would be to compare a polymer being read against a reference or consensus data, and to determine whether (a) the analysis should proceed; or (b) the polymer should be rejected from the sensor element as it is considered not of interest. In scenario (b) a new polymer should be introduced to the sensor element for analysis. Such a method involves analysing measurements taken from the polymer when it has partially translocated through the nanopore, i.e. during translocation of the polymer through the nanopore. In particular, the series of measurements taken from the polymer during the partial translocation are analysed using reference data derived from at least one reference sequence of polymer units. This analysis provides a measure of similarity between the sequence of polymer units of the partially translocated polymer and the at least one reference sequence. Responsive to that measure of similarity, action may be taken to reject the polymer to take measurements from a further polymer if the similarity to the reference sequence indicates no further analysis of the polymer is needed, for example because the polymer being measured is not of interest.

The rejection of the polymer allows measurements of a further polymer to be taken without completing the measurement of the polymer initially being measured. This provides a time saving in taking the measurements, because the action is taken “on-the-fly”, i.e. during the taking of measurements from a polymer. In typical applications, that time saving may be significant because biochemical analysis systems using nanopores can provide long continuous reads of polymers, whereas the analysis may identify at an early stage in such a read that no further measurements of the polymer currently being measured are needed. However, the use of this method can lead to device inefficiencies if, for instance, the polymer is not successfully rejected from the sensor element.

Device inefficiencies from a failure to reject the undesired polymer can manifest in a number of ways. In one example, the polymer is only partly rejected and the device continues to analyse the undesired polymer creating inaccurate data and using resources on the device (i.e. limited sensor elements, power, computational resource) to analyse unwanted data. In an alternative example, the undesired polymer may become blocked in the nanopore. In this scenario device resource can be inefficiently used when several unsuccessful attempts are used to unblock the nanopore, especially if the device is stuck in a reject/unblock feedback loop. Any continuous attempts to unblock a nanopore may use a relatively high voltage which is an unnecessary and inefficient use of power, and can unsettle or disturb neighbouring sensor elements in an array of sensor elements.

In a first aspect, the present invention provides an improved method of controlling a nanopore device for analysing a polymer, the nanopore device comprising at least one sensor element, the sensor element comprising a nanopore and a sensor, the method comprising: translocating a polymer through the nanopore; generating a series of measurements using the sensor as the polymer translocates through the nanopore; comparing the series of measurements to a reference data to determine a measurement of similarity; operating the nanopore device to eject the polymer from the nanopore if the measure of similarity is determined to be below a threshold value; and determining whether the polymer has been successfully ejected from the nanopore by analysing a further set of measurements taken by the sensor; wherein if it has been determined that the polymer has not been successfully ejected from the nanopore, the method further comprises operating the nanopore device to perform either: (i) At least one additional step to eject the polymer from the nanopore; or (ii) ceasing the taking of measurements from the nanopore.

In examples the predetermined value may be a measurement by the sensor when the nanopore is free from polymer. For example, the predetermined value is a configuration or measurement based on known properties of the nanopore device. In other words, a baseline measurement may be taken by the sensor element when the nanopore is known to be free of polymer. This may be taken, for example, before analyte or sample polymer is introduced to the nanopore device, or before analyte or sample polymer has been introduced into the sensor element(s). This is often referred to as open pore measurement, namely where there is an increased flux of ions through the pore due to the absence of polymer. This would, for example, give rise to an observed increased in current signal. Alternatively, the predetermined value may be a range of values, or a value that indicates a particular molecule, or chemical moiety, or an identifiable noise or pulse. In one such example the signal might be a bespoke and identifiable leader sequence of polymer units at the end of the polymer to be analysed. The nanopore device would be configured to recognise the signal measured from a leader sequence translocating through the nanopore.

Additionally or alternatively the predetermined value may be taken once a known polymer has fully translocated through a sensor. The predetermined value may be attributable to a baseline measurement for each sensor element, or taken from one sensor element but attributable to all sensor elements in an array device. Essentially, the measurement from the system is expected to be a particular value or in between the range of values to identify of an ejection had been successful.

In example, at least one sensor element may be operable to eject a polymer that is translocating through the nanopore. More specifically, the sensor may comprise an electrode, and the at least one sensor element is operable to eject a polymer that is translocating through the nanopore by application of an ejection bias voltage to eject the polymer, such that the step of operating the sensor element to eject the polymer from the nanopore is performed by applying an ejection bias voltage. The ejection bias voltage is provided to eject the polymer from the nanopore of the sensor element, which can be a full translocation through the nanopore, or a reverse translocation of a partially translocated polymer depending on the length of the polymer and the amount that has already translocated through the nanopore.

In examples the method may further comprise the additional step if the polymer has been successfully ejected: operating the sensor element to accept a further polymer to translocate through the nanopore, wherein operating the sensor element to accept a further polymer is performed by applying a translocation bias voltage sufficient to enable translocation of a further polymer therethrough. The improved method of the present invention has enabled the nanopore device to correctly determine that the sensor element is free from the rejected polymer and is ready to accept another polymer for analysis, thereby ensuring that the sensor elements of the device are used as efficiently as possible given the volume/number of polymers to analyse (i.e. reads of analyte), the length of polymers to be analysed, and the rate/speed at which polymers can be analysed by the nanopore device. The methods and devices of the prior art suffer from this inherent problem, namely that when ejecting or translocating particularly long strands of polynucleotides (such as ≥5 kb) the ejection step is not always successful. After a failed ejection measurements continue to be made on the existing polymer. The user is unclear whether these measurements are from a new strand or the existing stand since there is an assumption that all ejections are successful. In the event that a failed ejection occurs then the device is more inefficient since the measurements arising are further unwanted measurements from an undesired strand for analysis.

Conversely, the method comprises the additional step if the polymer has not been successfully ejected: the application of a second ejection bias voltage to eject the polymer, the second ejection bias voltage being higher than the first ejection bias voltage. In this scenario the improved method of the present invention has enabled the nanopore device to correctly determine that the sensor element is not free from the rejected polymer and is not ready to accept another polymer for analysis. The nanopore of the affected sensor element is determined to be blocked or has had administered an ejection bias voltage insufficient to fully eject the polymer (due to length, resistance in movement through the pore etc) and an increase in ejection bias voltage can be administered to attempt to unblock the pore. In this scenario the sensor elements of the device are used as efficiently as possible since they are not used to analyse polymer that has already been determined to be rejected.

Similarly in examples, the method may comprise the additional step if the polymer has not been successfully ejected after application of the second ejection bias voltage: the application of a third ejection bias voltage to eject the polymer, wherein the third ejection bias voltage is higher than the second ejection bias voltage. In this scenario the improved method of the present invention has enabled the nanopore device to correctly determine that the sensor element remains affected by the rejected polymer and is not ready to accept another polymer for analysis (and measurements from the currently affected sensor element should not be recorded or should be disregarded). The nanopore of the affected sensor element is still determined as blocked or has had administered an ejection bias voltage insufficient to fully eject the polymer (due to length, resistance in movement through the pore etc) and an increase in ejection bias voltage can be administered to attempt to unblock the pore. In this scenario the sensor elements of the device are used as efficiently as possible since they are not used to analyse polymer that has already been determined to be rejected.

In examples the nanopore device may comprise: a detection circuit comprising a plurality of detection channels each capable of taking electrical measurements from a sensor element, the number of sensor elements in the array being greater than the number of detection channels; and a switch arrangement capable of selectively connecting the detection channels to respective sensor elements in a multiplexed manner. In this regard the nanopore device can readily switch from receiving signal from an affected or blocked nanopore of a sensor element to receiving signal(s) from another sensor element(s) if required.

The nanopore device may determine to shut off or ignore signals being generated by this sensor element if it is determined to have not completed a successful ejection of a polymer at this stage. In examples if the polymer has not been successfully ejected then the method may further comprise, when operating the nanopore device to cease taking measurements from the currently selected sensor element.

In examples the reference data derived from at least one reference sequence of polymer units may represent actual or simulated measurements taken by a nanopore device, and said step of analysing the series of measurements taken from the polymer during the partial translocation comprises: comparing the series of measurements with the reference data. The reference data could relate to a part of a sequence of a polymer of interest. Alternatively, or additionally, the reference data could relate to a synthetic or tailored part or tag of a polymer of interest. The reference data is used to ensure that the polymer being analysed is a polymer of interest such that device resource is not used up analysing a signal generated by a polymer which is not of interest to the end user. The reference data can be a relatively short sequence so that when longer polymers are to be analysed the device can readily determine and reject polymers that are not of interest.

The rejection of the polymer allows measurements of a further polymer to be taken without completing the measurement of the polymer initially being measured. This provides a time saving in taking the measurements, because the action is taken “on-the-fly”, i.e. during the taking of measurements from a polymer. In typical applications, that time saving may be significant because biochemical analysis systems using nanopores can provide long continuous reads of polymers, whereas the analysis may identify at an early stage in such a read that no further measurements of the polymer currently being measured are needed.

For example in typical applications where the polymer is a polynucleotide, sequencing performed with 100% accuracy would allow an initial determination to be made after measurement of around 30 nucleotides. Thus, taking into account actually achievable accuracies, the determination may be made after measurement of a few hundred nucleotides, typically 250 nucleotides. This compares to nanopore device being able to perform measurements on sequences ranging in length from many hundreds to tens of thousands (and potentially more) nucleotides.

The reference data derived from at least one reference sequence of polymer units may represent a feature vector of time-ordered features representing characteristics of the measurements taken by a biochemical analysis system, and said step of analysing the series of measurements taken from the polymer during the partial translocation comprises: deriving, from the series of measurements, a feature vector of time-ordered features representing characteristics of the measurements, and comparing the derived feature vector with the reference data.

The reference data derived from at least one reference sequence of polymer units may represent the identity of the polymer units of the at least one reference sequence, and said step of analysing the series of measurements taken from the polymer during the partial translocation comprises: analysing the series of measurements to provide an estimate of the identity of the polymer units of a sequence of polymer units of the partially translocated polymer, and comparing the estimate with the reference data to provide the measure of similarity.

There are many techniques that can be used to interpret and resolve measurement signals from nanopore devices when estimated and determining the identity of polymer units. Two well know data processing techniques that are commonly used in this field are machine learning (such as modified neural networks) and probabilistic methods (such as an HMM involving k-mer analysis). Exemplary methods are disclosed in WO2013121224A1 and WO2018203084A1, which are herein incorporated by reference in their entirety.

It has been found that probabilistic methods such as HMM based k-mer modelling for measurement signal interpretation, although resource intensive and being computationally complex, offers a robust and easily trainable system for comparison of signal against a reference data. A review of probabilistic analysis tool being used in the field of nanopore devices can be found in the paper Martin et al, Genome Biology (2022) 23:11 which is herein incorporated by reference in its entirety.

In examples the measurements are dependent on a k-mer, being k polymer units of polymer, where k is an integer; the reference data represents a reference model that treats the measurements as observations of a reference series of k-mer states corresponding to the reference sequence of polymer units, wherein the reference model comprises: transition weightings for transitions between the k-mer states in the reference series of k-mer states; and in respect of each k-mer state, emission weightings for different measurements being observed when the k-mer state is observed, and said step of analysing the series of measurements taken from the polymer during the partial translocation comprises fitting the model to the series of the series of measurements to provide the measure of similarity as the fit of the model to the series of measurements.

In further examples the measurements may be dependent on a k-mer, being k polymer units of polymer, where k is an integer.

Such a method involves analysing measurements taken from the polymer when it has partially translocated through the nanopore, i.e. during translocation of the polymer through the nanopore. In particular, the series of measurements taken from the polymer during the partial translocation are analysed using reference data derived from at least one reference sequence of polymer units. This analysis provides a measure of fit to a model. Responsive to that measure of fit, action may be taken to reject the polymer and to take measurements from a further polymer, if the measure of fit indicates measurements are of poor quality.

The rejection of the polymer allows measurements of a further polymer to be taken without completing the measurement of the polymer initially being measured. This provides a time saving in taking the measurements, because the action is taken “on-the-fly”, i.e. during the taking of measurements from a polymer. In typical applications, that time saving may be significant because biochemical analysis systems using nanopores can provide long continuous reads of polymers, whereas the analysis may identify at an early stage that the measurements are of poor quality.

The nanopore may be a solid-state pore or a biological pore. In particular examples the nanopore may be a biological pore.

In examples the polymer is a polynucleotide, and the polymer units are nucleotides. The translocation of the polymer through the nanopore is performed in a ratcheted manner using, for instance a molecular motor or enzyme to control the rate of translocation. This at least ensures that there is an accurate and precise comparison between the polymer being analysed and the reference data.

In examples the nanopore device may comprise a sensor electrode and the measurements comprise electrical measurements. In particular examples the nanopore device may comprise a sensor electrode, and the measurements taken by the sensor are indicative of ion flow through the nanopore.

In a second aspect, the present invention provides a nanopore device for analysing polymers that comprise a sequence of polymer units, wherein the nanopore device comprises at least one sensor element that comprises a nanopore, a sensor and a data processor, and the nanopore device is operable to take successive measurements of a polymer from a sensor element, during translocation of the polymer through the nanopore of the sensor element, wherein the data processor of the nanopore device is arranged, when a polymer has partially translocated through the nanopore, to analyse the series of measurements taken from the polymer during the partial translocation thereof, comparing the series of measurements to a reference data to determine a measurement of similarity; wherein the data processor of the nanopore device is further arranged, responsive to the measure of similarity, to eject the polymer and determine whether the polymer has been successfully ejected from the nanopore by analysing a further set of measurements taken by the sensor.

In a third aspect, the present invention provides a method of controlling a nanopore device for analysing polymers that comprise a sequence of polymer units, wherein the nanopore device comprises at least one sensor element that comprises a nanopore and a sensor, and the nanopore device is operable to take successive measurements of a polymer from a sensor element, during translocation of the polymer through the nanopore of the sensor element, and the nanopore device is operable to generate successive measurements of a polymer from a sensor element, during translocation of the polymer through the nanopore of the sensor element, wherein the method comprises, when a polymer has partially translocated through the nanopore, analysing the series of measurements taken from the polymer during the partial translocation thereof by deriving a measure of fit to a model that treats the measurements as observations of a series of k-mer states of different possible types and comprises: transition weightings, in respect of each transition between successive k-mer states in the series of k-mer states, for possible transitions between the possible types of k-mer state; and emission weightings, in respect of each type of k-mer state that represent the chances of observing given values of measurements for that k-mer, and responsive to the measure of fit, operating the biochemical analysis system to eject the polymer and determine whether the polymer has been successfully ejected from the nanopore by analysing a further set of measurements taken by the sensor.

In a forth aspect, the present invention provides a nanopore device for analysing polymers that comprise a sequence of polymer units, wherein the nanopore device comprises at least one sensor element that comprises a nanopore and a sensor, and the nanopore device is operable to take successive measurements of a polymer from a sensor element, during translocation of the polymer through the nanopore of the sensor element, wherein the biochemical analysis system is arranged, when a polymer has partially translocated through the nanopore, analysing the series of measurements taken from the polymer during the partial translocation thereof by deriving a measure of fit to a model that treats the measurements as observations of a series of k-mer states of different possible types and comprises: transition weightings, in respect of each transition between successive k-mer states in the series of k-mer states, for possible transitions between the possible types of k-mer state; and emission weightings, in respect of each type of k-mer state that represent the chances of observing given values of measurements for that k-mer, and the biochemical analysis system is arranged, responsive to the measure of fit, to eject the polymer and determine whether the polymer has been successfully ejected from the nanopore by analysing a further set of measurements taken by the sensor.

To allow better understanding, embodiments of the present invention will now be described by way of non-limitative example with reference to the accompanying drawings, in which:

FIG. 1 is a schematic diagram of a nanopore device;

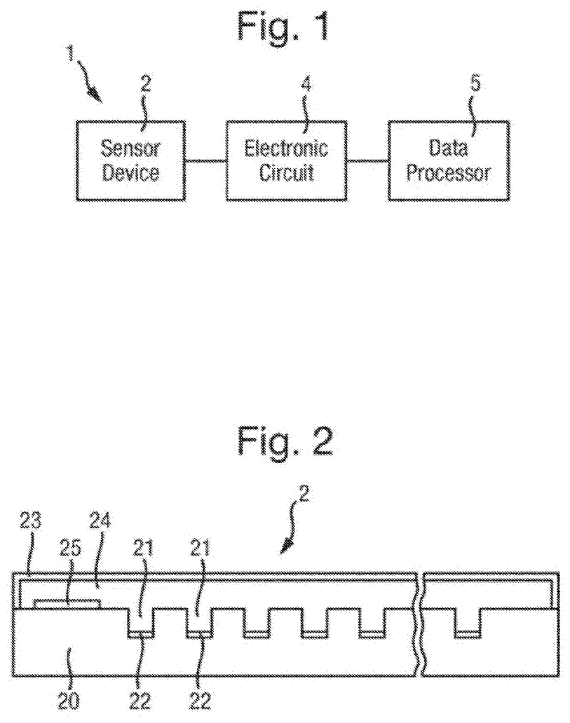

FIG. 2 is a cross-sectional view of a nanopore sensor device;

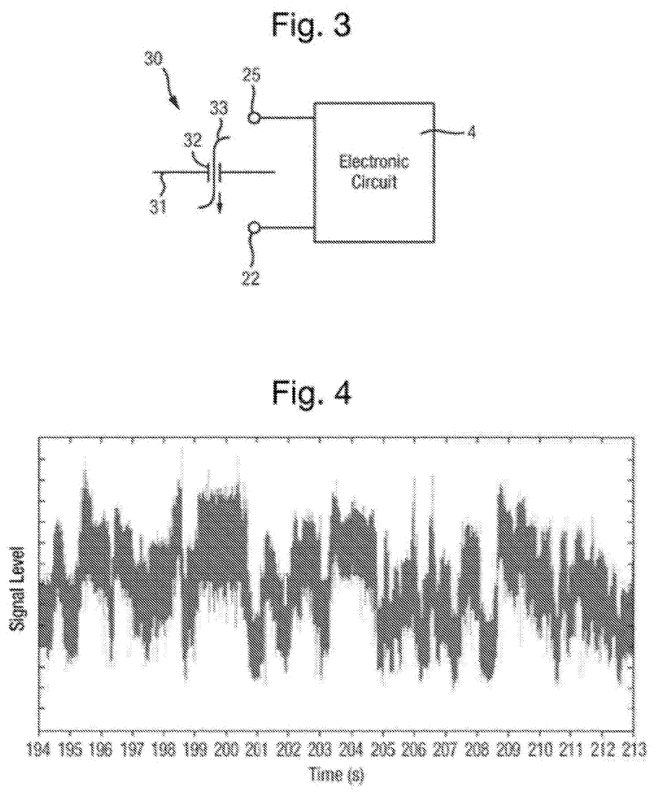

FIG. 3 is a schematic view of a sensor element of the nanopore device;

FIG. 4 is a plot of a typical signal trace of an event measured over time by a sensor element;

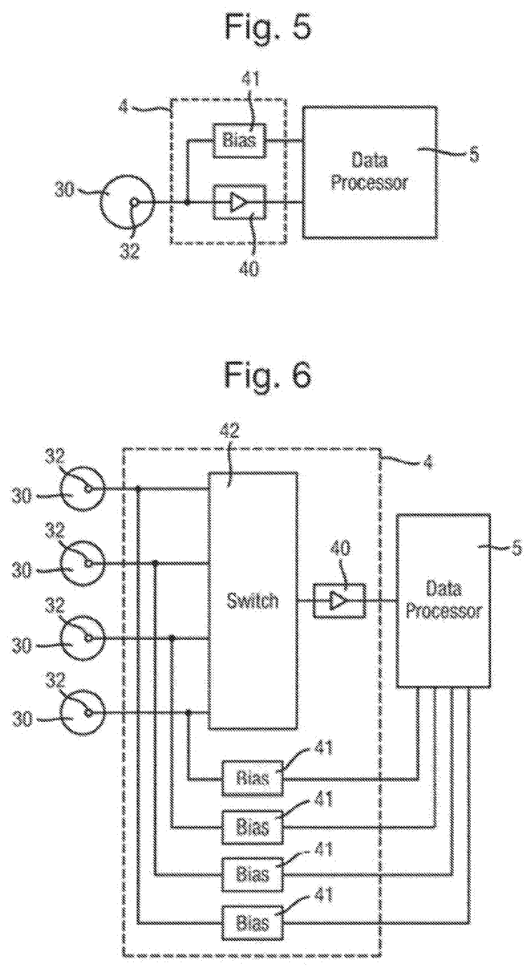

FIG. 5 is a diagram of the electronic circuit of a sensor element;

FIG. 6 is a diagram of the electronic circuit of an array of sensor elements;

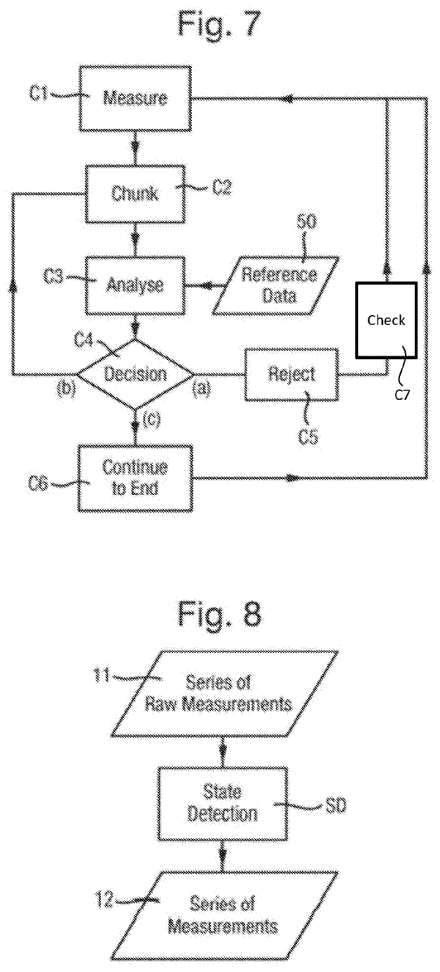

FIG. 7 is a flow chart of a method of controlling the nanopore device to analyse polymers;

FIG. 8 is a flow chart of a state detection step;

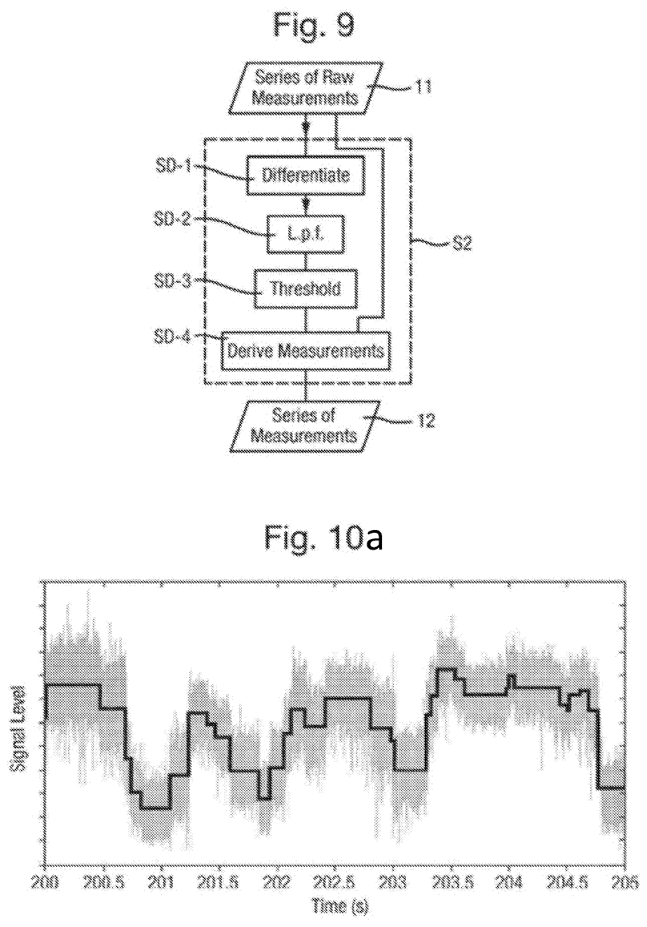

FIG. 9 is a detailed flow chart of an example of the state detection step;

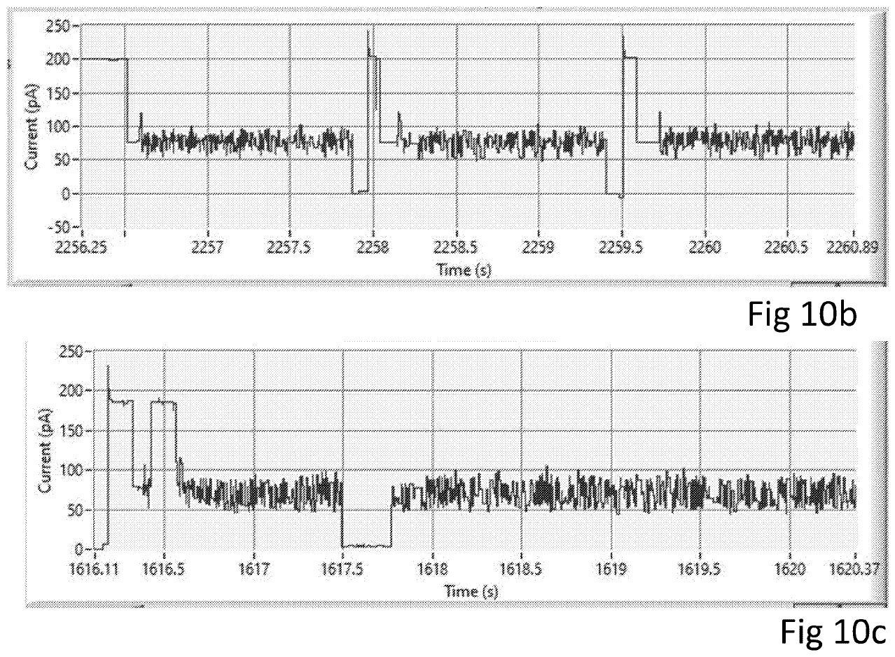

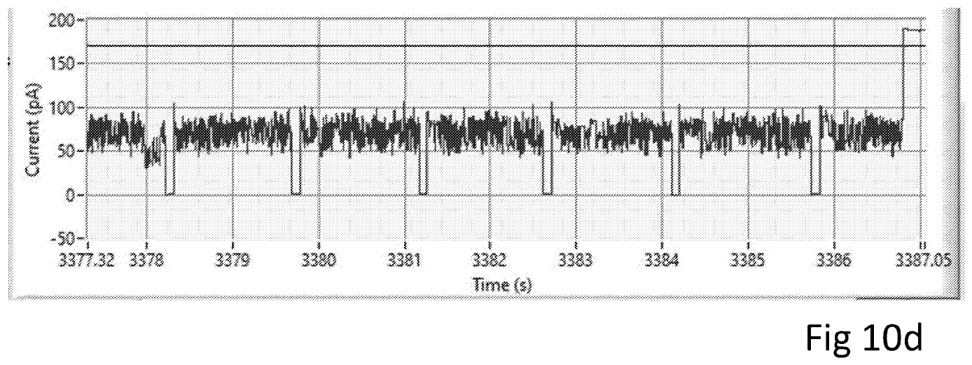

FIG. 10a is a plot of a series of raw measurements subject to the state detection step and of the resultant series of measurements;

FIG. 10b is a plot of an atypical signal trace of an event measured over time by a sensor element where successful ejections of the polymer from the nanopore are followed by a new strand capture;

FIG. 10c is a plot of an atypical signal trace of an event measured over time by a sensor element where there are multiple unsuccessful attempts to eject the polymer from the nanopore followed by a natural end to the translocation of the polymer;

FIG. 10d is a plot of an atypical signal trace of an event measured over time by a sensor element where there a single unsuccessful attempt to eject the polymer from the nanopore and the polymer is left to translocate through the nanopore;

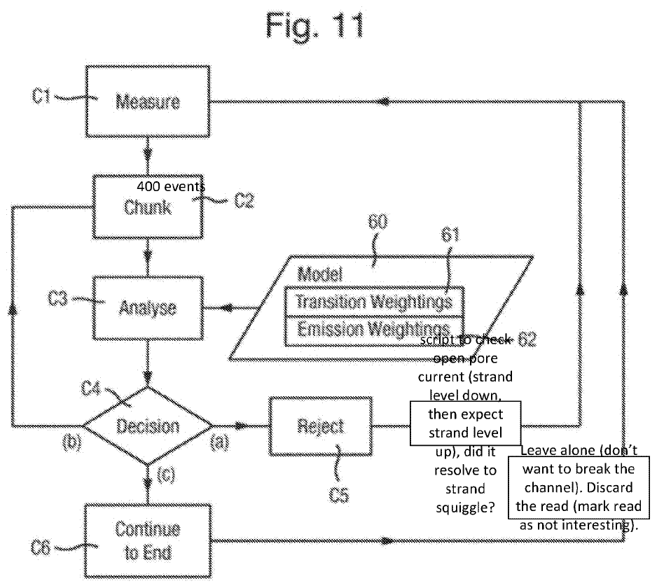

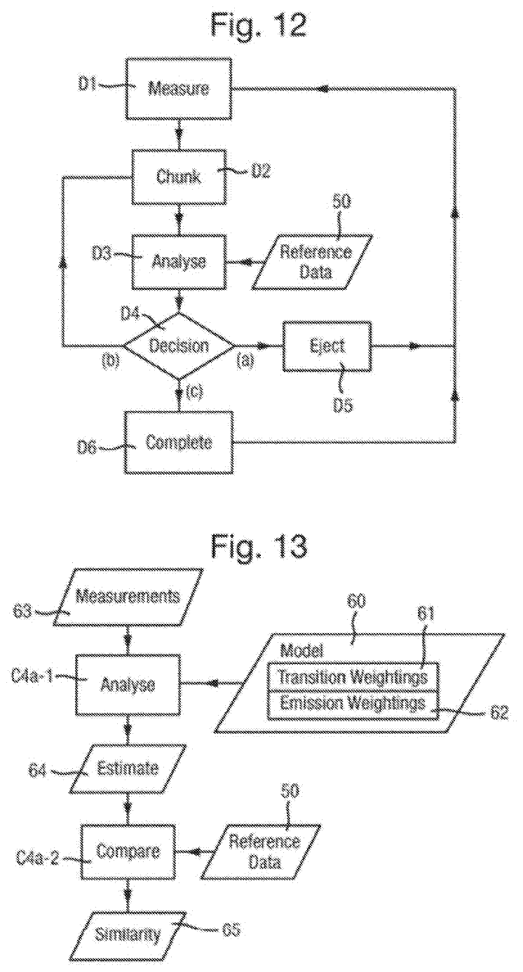

FIGS. 11 and 12 are flow charts of methods of controlling the biochemical analysis system;

FIGS. 13 to 16 are flow charts of different methods for analysing reference data of different forms;

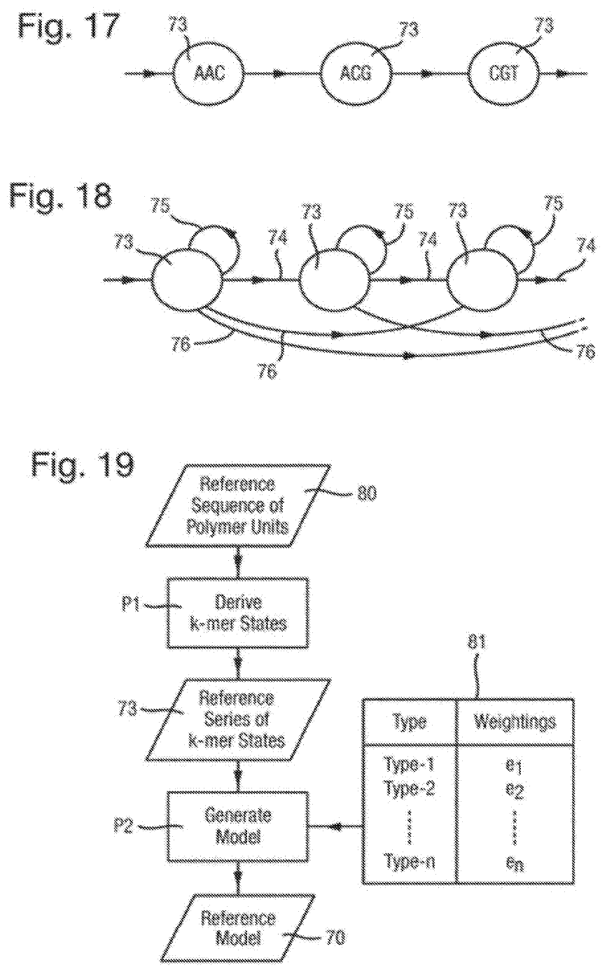

FIG. 17 is a state diagram of an example of a reference series of k-mer states;

FIG. 18 is a state diagram of a reference series of k-mer states illustrating possible types of transition between the k-mer states;

FIG. 19 is a flow chart of a first process for generating a reference model;

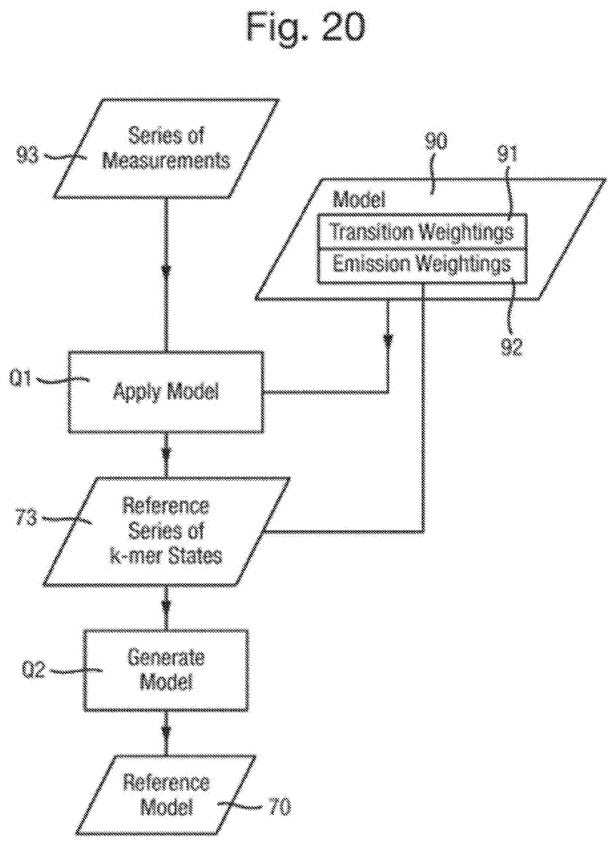

FIG. 20 is a flow chart of a first process for generating a reference model;

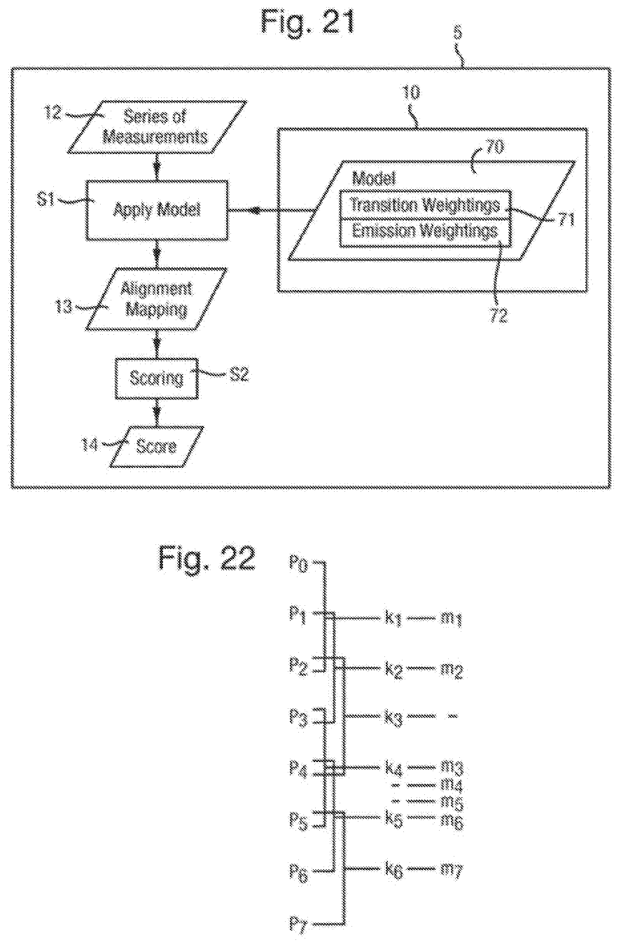

FIG. 21 is a flow chart of a method of estimating an alignment mapping; and

FIG. 22 is a diagram of an alignment mapping.

The various features described below are examples and not limitative. Also, the features described are not necessarily applied together and may be applied in any combination.

FIG. 1 illustrates a nanopore device 1 for analysing polymers. There will first be described the nature of the polymer that are analysed.

The polymer comprises a sequence of polymer units. Each given polymer unit may be of different types (or identities), depending on the nature of the polymer.

The polymer may be a polynucleotide (or nucleic acid), a polypeptide such as a protein, a polysaccharide, oligosaccharide, or any other polymer. The polymer may be natural or synthetic. The polymer units may be nucleotides. The nucleotides may be of different types that include different nucleobases.

The polynucleotide may be deoxyribonucleic acid (DNA), ribonucleic acid (RNA), cDNA or a synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains. The polynucleotide may be single-stranded, be double-stranded or comprise both single-stranded and double-stranded regions. Typically cDNA, RNA, GNA, TNA or LNA are single stranded.

The methods described herein may be used to identify any nucleotide. The nucleotide can be naturally occurring or artificial. A nucleotide typically contains a nucleobase (which may be shortened herein to “base”), a sugar and at least one phosphate group. The nucleobase is typically heterocyclic. Suitable nucleobases include purines and pyrimidines and more specifically adenine, guanine, thymine, uracil and cytosine. The sugar is typically a pentose sugar. Suitable sugars include, but are not limited to, ribose and deoxyribose. The nucleotide is typically a ribonucleotide or deoxyribonucleotide. The nucleotide typically contains a monophosphate, diphosphate or triphosphate.

The nucleotide can include a damaged or epigenetic base. The nucleotide can be labelled or modified to act as a marker with a distinct signal. This technique can be used to identify the absence of a base, for example, an abasic unit or spacer in the polynucleotide.

Of particular use when considering measurements of modified or damaged DNA (or similar systems) are the methods where complementary data are considered. The additional information provided allows distinction between a larger number of underlying states.

The polymer may also be a type of polymer other than a polynucleotide, some non-limitative examples of which are as follows.

The polymer may be a polypeptide, in which case the polymer units may be amino acids that are naturally occurring or synthetic.

The polymer may be a polysaccharide, in which case the polymer units may be monosaccharides.

The polymer may comprise any length of polymer units. In the case of translocation of polynucleotides through a nanopore, the length may range between 5 kB and 4 MB or greater. The present inventors have observed when translocating polynucleotides from the cis side of the nanopore to the trans side, it can be difficult to eject the polymer from the nanopore when a substantial length of the polynucleotide has already exited the nanopore on the trans side and a one or more further voltage biases are required to eject the polynucleotide from the nanopore. The method of the invention is thus particularly beneficial when the ejection step is carried out for polymers wherein at least 50 kB, 100 kB, 500 kB or 1 MB of the polymer such as a polynucleotide has already translocated the nanopore.

Herein, the term ‘k-mer’ refers to a group of k-polymer units, where k is a positive integer, including the case that k is one, in which the k-mer is a single polymer unit. In some contexts, reference is made to k-mers where k is a plural integer, being a subset of k-mers in general excluding the case that k is one.

Each given k-mer may therefore also be of different types, corresponding to different combinations of the different types of each polymer unit of the k-mer.

Reverting to FIG. 1, the nanopore device 1 comprises a sensor device 2 connected to an electronic circuit 4 which is in turn connected to a data processor 6.

There will first be described some examples in which the sensor device 2 comprises an array of sensor elements that each comprise a biological nanopore.

In a first form, the sensor device 2 may have a construction as shown in cross-section in FIG. 2 comprising a body 20 in which there is formed an array of wells 21 each being a recess having a sensor electrode 22 arranged therein. A large number of wells 21 is provided to optimise the data collection rate of the apparatus 1. In general, there may be any number of wells 21, typically 256 or 1024, although only a few of the wells 21 are shown in FIG. 2. The body 20 is covered by a cover 23 that extends over the body 20 and is hollow to define a chamber 24 into which each of the wells 21 opens. A common electrode 25 is disposed within the chamber 23. In this first form, the sensor device 2 may be an apparatus as described in further detail in WO 2009/077734, the teachings of which may be applied to the nanopore device 1, and which is incorporated herein by reference.

In a second form, the sensor device 2 may have a construction as described in detail in WO 2014/064443, the teachings of which may be applied to the nanopore device 1, and which is incorporated herein by reference. In this second form, the sensor device 2 has a generally similar configuration to the first form, including an array of compartments which are generally similar to the wells 21 although they have a more complicated construction and which each contain a sensor electrode 22.

The sensor device 2 is prepared to form an array of sensor elements 30, one of which is shown schematically in FIG. 3. Each sensor element 30 is made by forming a membrane 31 across a respective well 21 in the first form of the sensor device 2 or across each compartment in the second form of the sensor device 2, and then by inserting a pore 32 into the membrane 31. The membrane 31 may be made of amphiphilic molecules such as lipid. The pore 32 is a biological nanopore. This preparation may be performed for the first form of the sensor device 2 using the techniques and materials described in detail in WO 2009/077734, or for the second form of the sensor device 2 using the techniques and materials described in detail in WO 2014/064443.

Each sensor element 30 is capable of being operated to take electrical measurements from a polymer during translocation of the polymer 33 through the pore 32, using the sensor electrode 22 in respect of each sensor element 30 and the common electrode 25. The translocation of the polymer 33 through the pore 32 generates a characteristic signal in the measured property that may be observed and may be referred to overall as an “event”.

The nanopore channel is a pore 32, typically having a size of the order of nanometres. In embodiments where the molecular entities are polymers that interact with the nanopore channel 32 while translocating therethrough in which case the nanopore channel 32 is of a suitable size to allow the passage of polymers therethrough.

The nanopore may be a protein pore or a solid-state pore. The dimensions of the pore may be such that only one polymer may translocate the pore at a time.

Where the nanopore is a protein pore, it may have the following properties.

The nanopore may be a transmembrane protein pore. Transmembrane protein pores for use in accordance with the invention include, but are not limited to, β-toxins, such as α-hemolysin, anthrax toxin and leukocidins, and outer membrane proteins/porins of bacteria, such as Mycobacterium smegmatis porin (Msp), for example MspA, lysenin, outer membrane porin F (OmpF), outer membrane porin G (OmpG), outer membrane phospholipase A and Neisseria autotransporter lipoprotein (NalP). α-helix bundle pores comprise a barrel or channel that is formed from α-helices. Suitable α-helix bundle pores include, but are not limited to, inner membrane proteins and α outer membrane proteins, such as WZA and ClyA toxin. The transmembrane pore may be derived from lysenin. The pore may be derived from CsgG, such as disclosed in WO-2016/034591, WO-2017/149316, WO-2017/149317, WO-2017/149318 or WO-2019/002893 all of which are herein incorporated by reference in their entirety. The pore may be a DNA origami pore.

The protein pore may be a naturally occurring pore or may be a mutant pore. The pore may be fully synthetic.

Where the nanopore is a protein pore, it may be inserted into a membrane that is supported in the sensor element 30. Such a membrane may be an amphiphilic layer, for example a lipid bilayer. An amphiphilic layer is a layer formed from amphiphilic molecules, such as phospholipids, which have both hydrophilic and lipophilic properties. The amphiphilic layer may be a monolayer or a bilayer. The amphiphilic layer may be a co-block polymer such as disclosed in WO 2014/064444. Alternatively, a protein pore may be inserted into an aperture provided in a solid-state layer, for example as disclosed in WO 2012/005857.

The nanopore may comprise an aperture formed in a solid-state layer, which may be referred to as a solid-state pore. The aperture may be a well, gap, channel, trench or slit provided in the solid-state layer along or into which analyte may pass. Solid-state layers can be formed from both organic and inorganic materials including, but not limited to, microelectronic materials, insulating materials such as Si3N4, Al2O3, and SiO, organic and inorganic polymers such as polyamide, plastics such as Teflon® or elastomers such as two-component addition-cure silicone rubber, and glasses. The solid-state layer may be formed from graphene.

Molecular entities interact with the nanopores in the sensing elements 30 causing output an electrical signal at the electrode 31 that is dependent on that interaction.

In one type of sensor device 2, the electrical signal may be the ion current flowing through the nanopore. Similarly, electrical properties other than ion current may be measured. Some examples of alternative types of property include without limitation: ionic current, impedance, a tunnelling property, for example tunnelling current (for example as disclosed in Ivanov A P et al., Nano Lett. 2011 Jan. 12; 11(1):279-85 which is herein incorporated by reference in its entirety), and a FET (field effect transistor) voltage (for example as disclosed in WO2005/124888 which is herein incorporated by reference in its entirety). One or more optical properties may be used, optionally combined with electrical properties (Soni G V et al., Rev Sci Instrum. 2010 January; 81(1):014301 which is herein incorporated by reference in its entirety). The property may be a transmembrane current, such as ion current flow through a nanopore. The ion current may typically be the DC ion current, although in principle an alternative is to use the AC current flow (i.e. the magnitude of the AC current flowing under application of an AC voltage).

The interaction may occur during translocation of the molecular entities with respect to the nanopore, for example through the nanopore.

The electrical signal provides as series of measurements of a property that is associated with an interaction between the molecular entity and the nanopore. Such an interaction may occur at a constricted region of the nanopore. For example in the case that the molecular entity is a polymer comprising a series of polymer units which translocate with respect to the nanopore, the measurements may be of a property that depends on the successive polymer units translocating with respect to the pore.

Ionic solutions may be provided on either side of the nanopore. A sample containing the molecular entities of interest that are polymers may be added to one side of the nanopore, for example in the sample chamber wells 21 in the sensor device of FIG. 2. A nanopore is provided in the membrane 31 and allowed to translocate with respect to the nanopore 32, for example under a potential difference or chemical gradient. The electrical signal may be derived during the translocation of the polymer with respect to the pore, for example taken during translocation of the polymer 33 through the nanopore 32. The polymer 33 may partially translocate with respect to the nanopore 32.

In order to allow measurements to be taken as a polymer 33 translocates through a nanopore, the rate of translocation can be controlled by a binding moiety that binds to the polymer 33. Typically the binding moiety can move a polymer through the nanopore with or against an applied field. The binding moiety can be a molecular motor using for example, in the case where the binding moiety is an enzyme, enzymatic activity, or as a molecular brake. Where the polymer is a polynucleotide there are a number of methods proposed for controlling the rate of translocation including use of polynucleotide binding enzymes. Suitable enzymes for controlling the rate of translocation of polynucleotides include, but are not limited to, polymerases, translocases, helicases, exonucleases, single stranded and double stranded binding proteins, and topoisomerases, such as gyrases. For other polymer types, binding moieties that interact with that polymer type can be used. The binding moiety may be any disclosed in WO-2010/086603, WO-2012/107778, and Lieberman K R et al, J Am Chem Soc. 2010; 132(50):17961-72), and for voltage gated schemes (Luan B et al., Phys Rev Lett. 2010; 104(23):238103) which are all herein incorporated by reference in their entireties.

The binding moiety can be used in a number of ways to control the polymer motion. The binding moiety can move the polymer through the nanopore with or against the applied field. The binding moiety can be used as a molecular motor using for example, in the case where the binding moiety is an enzyme, enzymatic activity, or as a molecular brake. The translocation of the polymer may be controlled by a molecular ratchet that controls the movement of the polymer through the pore. The molecular ratchet may be a polymer binding protein.

The polynucleotide handling enzyme may be for example one of the types of polynucleotide handling enzyme described in WO 2015/140535, WO2015/055981 or WO-2010/086603.

Translocation of the polymer 33 through the nanopore 32 may occur, either cis to trans or trans to cis, either with or against an applied potential. The translocation may occur under an applied potential which may control the translocation.

Exonucleases that act progressively or processively on double stranded DNA can be used on the cis side of the pore to feed the remaining single strand through under an applied potential or the trans side under a reverse potential. Likewise, a helicase that unwinds the double stranded DNA can also be used in a similar manner. There are also possibilities for sequencing applications that require strand translocation against an applied potential, but the DNA must be first “caught” by the enzyme under a reverse or no potential. With the potential then switched back following binding the strand will pass cis to trans through the pore and be held in an extended conformation by the current flow. The single strand DNA exonucleases or single strand DNA dependent polymerases can act as molecular motors to pull the recently translocated single strand back through the pore in a controlled stepwise manner, trans to cis, against the applied potential. Alternatively, the single strand DNA dependent polymerases can act as a molecular brake slowing down the movement of a polynucleotide through the pore. Any moieties, techniques or enzymes described in WO-2012/107778 or WO-2012/033524 which are both herein incorporated by reference in their entireties could be used to control polymer motion.

Control of translocation of the polymer analyte through the nanopore may be carried out by other methods, such as the use of a clamp and the application of a translocation force as disclosed in WO 2019/006214. Other forces to control translocation can be alternatively employed, including hydrostatic pressure, voltage control, the forces of optical or magnetic fields, and combinations of the above, such as disclosed Lu et al, Nanoletters 13:3048-3052, 2013 and Keyser et al, Nature Physics, 2:473-477, 2008.

The sensing elements 30 and/or the molecular entities may be adapted to capture molecular entities within a vicinity of the respective nanopores. For example sensing elements 30 may further comprise capture moieties arranged to capture molecular entities within a vicinity of the respective nanopores. The capture moieties may be any of the binding moieties or exonucleases described above with also have the purpose of controlling the translocation or may be separately provided.

The capture moieties may be attached to the nanopores of the sensing elements. At least one capture moiety may be attached to the nanopore of each sensor element.

The capture moiety may be a tag or tether which binds to the molecular entities. In that case the molecular entity may be adapted to achieve that binding.

Such a tag or tether may be attached to the nanopore, for example as disclosed in WO 2018/100370 which is herein incorporated by reference in its entirety, and as further described herein below.

Alternatively in a case the nanopore is inserted in a membrane, such a tag or tether may be attached to the membrane, for example as disclosed in WO 2012/164270 which is herein incorporated by reference in its entirety.

The methods described herein may comprise the use of adapters attached to the molecular entity to be determined such as a polynucleotide, for the purpose of capturing them in the nanopore. By way of example, polynucleotide adapters suitable for use in nanopore sequencing of polynucleotides are known in the art. Adapters for use in nanopore sequencing of polynucleotides may comprise at least one single stranded polynucleotide or non-polynucleotide region. For example, Y-adapters for use in nanopore sequencing are known in the art. A Y adapter typically comprises (a) a double stranded region and (b) a single stranded region or a region that is not complementary at the other end. A Y adapter may be described as having an overhang if it comprises a single stranded region. The presence of a non-complementary region in the Y adapter gives the adapter its Y shape since the two strands typically do not hybridise to each other unlike the double stranded portion. The Y adapter may comprise one or more anchors.

The Y adapter preferably comprises a leader sequence which preferentially threads into the pore. The leader sequence typically comprises a polymer. The polymer is preferably negatively charged. The polymer is preferably a polynucleotide, such as DNA or RNA, a modified polynucleotide (such as abasic DNA), PNA, LNA, polyethylene glycol (PEG) or a polypeptide. The leader preferably comprises a polynucleotide and more preferably comprises a single stranded polynucleotide. The adapter may be ligated to a polymer analyte using any method known in the art. The leader sequence may give rise to a recognisably different signal pattern or measurement on the signal trace compared to the signal generated by polymers to be analysed, such that it can be used to determine if a new polymer is beginning to translocate through the pore.

The analyte may comprise a membrane anchor or a transmembrane pore anchor to attach the analyte to the membrane. For example, a membrane anchor or transmembrane pore anchor may promote localisation of the adapter and coupled polynucleotide within a vicinity of the nanopore. The anchor may be a polypeptide anchor and/or a hydrophobic anchor that can be inserted into the membrane. In one embodiment, the hydrophobic anchor is a lipid, fatty acid, sterol, carbon nanotube, polypeptide, protein or amino acid, for example cholesterol, palmitate or tocopherol.

The anchor may comprise a linker, or 2, 3, 4 or more linkers. Preferred linkers include, but are not limited to, polymers, such as polynucleotides, polyethylene glycols (PEGs), polysaccharides and polypeptides. These linkers may be linear, branched or circular. Suitable linkers are described in WO 2010/086602. Examples of suitable anchors and methods of attaching anchors to adapters are disclosed in WO 2012/164270 and WO 2015/150786 which are both herein incorporated by reference in their entireties.

Examples of tags and tethers which are attached to the nanopore are as follows.

Nanopores for use in the methods described herein may be modified to comprise one or more binding sites for binding to one or more analytes (e.g. molecular entities) and thereby acting as a capture moiety. In some embodiments, the nanopores may be modified to comprise one or more binding sites for binding to an adaptor attached to the analytes. For example, in some embodiments, the nanopores may bind to a leader sequence of the adaptor attached to the analytes. In some embodiments, the nanopores may bind to a single stranded sequence in the adaptor attached to the analytes.

In some embodiments, the nanopores are modified to comprise one or more tags or tethers, each tag or tether comprising a binding site for the analyte. In some embodiments, the nanopores are modified to comprise one tag or tether per nanopore, each tag or tether comprising a binding site for the analyte.

In some embodiments, the tag or tether may comprise or be an oligonucleotide.

Other examples of a tag or tether include, but are not limited to His tags, biotin or streptavidin, antibodies that bind to analytes, aptamers that bind to analytes, analyte binding domains such as DNA binding domains (including, e.g., peptide zippers such as leucine zippers, single-stranded DNA binding proteins (SSB)), and any combinations thereof.

The tag or tether may be attached to the external surface of the nanopore, e.g., on the cis side of a membrane, using any methods known in the art. For example, one or more tags or tethers can be attached to the nanopore via one or more cysteines (cysteine linkage), one or more primary amines such as lysines, one or more non-natural amino acids, one or more histidines (His tags), one or more biotin or streptavidin, one or more antibody-based tags, one or more enzyme modification of an epitope (including, e.g., acetyl transferase), and any combinations thereof. Suitable methods for carrying out such modifications are well-known in the art. Suitable non-natural amino acids include, but are not limited to, 4-azido-L-phenylalanine (Faz) and any one of the amino acids numbered 1-71 in FIG. 1 of Liu C. C. and Schultz P. G., Annu. Rev. Biochem., 2010, 79, 413-444 which is herein incorporated by reference in its entirety.

In some embodiments where one or more tags or tethers are attached to the nanopore via cysteine linkage(s), the one or more cysteines can be introduced to one or more monomers that form the nanopore by substitution.

The transmembrane pore may be modified to enhance capture of polynucleotides. For example, the pore may be modified to increase the positive charges within the entrance to the pore and/or within the barrel of the pore. Such modifications are known in the art. For example, WO 2010/055307 discloses mutations in a-hemolysin that increase positive charge within the barrel of the pore.

Modified MspA, lysenin and CsgG pores comprising mutations that enhance polynucleotide capture are disclosed in WO 2012/107778, WO 2013/153359 and WO 2016/034591, respectively which are all herein incorporated by reference in their entireties. Any of the modified pores disclosed in these publications may be used herein.

In general, when the measurement is current measurement of ion current flow through the pore 32, the ion current may typically be the DC ion current, although in principle an alternative is to use the AC current flow (i.e. the magnitude of the AC current flowing under application of an AC voltage).

The nanopore device 1 may take electrical measurements of types other than current measurements of ion current through a nanopore as described above.

Other possible electrical measurement include: current measurements, impedance measurements, tunnelling measurements (for example as disclosed in Ivanov A P et al., Nano Lett. 2011 Jan. 12; 11(1):279-85), and field effect transistor (FET) measurements (for example as disclosed in WO2005/124888).

As an alternative to electrical measurements, the nanopore device 1 may take optical measurements. A suitable optical method involving the measurement of fluorescence is disclosed by J. Am. Chem. Soc. 2009, 131 1652-1653.

Optical measurements may be combined with electrical measurements (Soni G V et al., Rev Sci Instrum. 2010 January; 81(1):014301).

The nanopore device 1 may take simultaneous measurements of different natures. The measurement may be of different natures because they are measurements of different physical properties, which may be any of those described above. Alternatively, the measurements may be of different natures because they are measurements of the same physical properties but under different conditions, for example electrical measurements such as current measurements under different bias voltages.

Typically, each measurement taken by the nanopore device 1 is dependent on a k-mer, being k polymer units of the respective sequence of polymer units, where k is a positive integer. Although ideally the measurements would be dependent on a single polymer unit (i.e. where k is one), with many typical types of the nanopore device 1, each measurement is dependent on a k-mer of plural polymer units (i.e. where k is a plural integer). That is, each measurement is dependent on the sequence of each of the polymer units in the k-mer where k is a plural integer.

In a series of measurements taken by the nanopore device 1, successive groups of plural measurements are dependent on the same k-mer. The plural measurements in each group are of a constant value, subject to some variance discussed below, and therefore form a “level” in a series of raw measurements. Such a level may typically be formed by the measurements being dependent on the same k-mer (or successive k-mers of the same type) and hence correspond to a common state of the nanopore device 1.

The signal moves between a set of levels, which may be a large set. Given the sampling rate of the instrumentation and the noise on the signal, the transitions between levels can be considered instantaneous, thus the signal can be approximated by an idealised step trace.

The measurements corresponding to each state are constant over the time scale of the event, but for most types of the nanopore device 1 will be subject to variance over a short time scale. Variance can result from measurement noise, for example arising from the electrical circuits and signal processing, notably from the amplifier in the particular case of electrophysiology. Such measurement noise is inevitable due the small magnitude of the properties being measured. Variance can also result from inherent variation or spread in the underlying physical or biological system of the nanopore device 1. Most types of the nanopore device 1 will experience such inherent variation to greater or lesser extents. For any given types of the nanopore device 1, both sources of variation may contribute or one of these noise sources may be dominant.

In addition, typically there is no a priori knowledge of number of measurements in the group, this varying unpredictably.

These two factors of variance and lack of knowledge of the number of measurements can make it hard to distinguish some of the groups, for example where the group is short and/or the levels of the measurements of two successive groups are close to one another.

The series of raw measurements may take this form as a result of the physical or biological processes occurring in the nanopore device 1. Thus, in some contexts each group of measurements may be referred to as a “state”.

For example, in some types of the nanopore device 1, the event consisting of translocation of the polymer through the pore 32 may occur in a ratcheted manner. During each step of the ratcheted movement, the ion current flowing through the nanopore at a given voltage across the pore 32 is constant, subject to the variance discussed above. Thus, each group of measurements is associated with a step of the ratcheted movement. Each step corresponds to a state in which the polymer is in a respective position relative to the pore 32. Although there may be some variation in the precise position during the period of a state, there are large scale movements of the polymer between states. Depending on the nature of the nanopore device 1, the states may occur as a result of a binding event in the nanopore.

The duration of individual states may be dependent upon a number of factors, such as the potential applied across the pore, the type of enzyme used to ratchet the polymer, whether the polymer is being pushed or pulled through the pore by the enzyme, pH, salt concentration and the type of nucleoside triphosphate present. The duration of a state may vary typically between 0.5 ms and 3s, depending on the nanopore device 1, and for any given nanopore system, having some random variation between states. The expected distribution of durations may be determined experimentally for any given nanopore device 1.

The extent to which a given nanopore device 1 provides measurements that are dependent on k-mers and the size of the k-mers may be examined experimentally. Possible approaches to this are disclosed in WO-2013/041878.

Reverting to the nanopore device 1 may take electrical measurements of types other than current measurements of ion current through a nanopore as described above.

Other possible electrical measurement include: current measurements, impedance measurements, tunnelling measurements (for example as disclosed in Ivanov A P et al., Nano Lett. 2011 Jan. 12; 11(1):279-85), and field effect transistor (FET) measurements (for example as disclosed in WO2005/124888 which is herein incorporated by reference in its entirety).

Reverting to FIG. 1, the arrangement of the electronic circuit 4 will now be discussed. The electronic circuit 4 is connected to the sensor electrode 22 in respect of each sensor element 30 and to the common electrode 25. The electronic circuit 4 may have an overall arrangement as described in WO 2011/067559 which is herein incorporated by reference in its entirety. The electronic circuit 4 is arranged as follows to control the application of bias voltages across each sensor element 3 and to take the measurements from each sensor element 3.

An arrangement for the electronic circuit 4 is illustrated in FIG. 5 which shows components in respect of a single sensor element 30 that are replicated for each one of the sensor elements 30. In this arrangement, the electronic circuit 4 includes a detection channel 40 and a bias control circuit 41 each connected to the sensor electrode 22 of the sensor element 30.

The detection channel 40 takes measurements from the sensor electrode 22. The detection channel 40 is arranged to amplify the electrical signals from the sensor electrode 22. The detection channel 40 is therefore designed to amplify very small currents with sufficient resolution to detect the characteristic changes caused by the interaction of interest. The detection channel 40 is also designed with a sufficiently high bandwidth to provide the time resolution needed to detect each such interaction. These constraints require sensitive and therefore expensive components. Specifically, the detection channel 40 may be arranged as described in detail in WO 2010/122293 or WO 2011/067559 to each of which reference is made and each of which is incorporated herein by reference.

The bias control circuit 41 supplies a bias voltage to the sensor electrode 22 for biasing the sensor electrode 22 with respect to the input of the detection channel 40.

During normal operation, the bias voltage supplied by the bias control circuit 41 is selected to enable translocation of a polymer through the pore 32. Such a bias voltage may typically be of a level up to −200 mV.

The bias voltage supplied by the bias control circuit 41 may also be selected so that it is sufficient to eject the translocating from the pore 32. By causing the bias control circuit 41 to supply such a bias voltage, the sensor element 30 is operable to eject a polymer that is translocating through the pore 32. To ensure reliable ejection, the bias voltage is typically a reverse bias, although that is not always essential. When this bias voltage is applied, the input to the detection circuit 40 is designed to remain at a constant bias potential even when presented with a negative current (of similar magnitude to the normal current, typically of magnitude 50 pA to 100 pA).

A typical signal trace of an event measured over time by a sensor element is shown in FIG. 4. Fluctuations in signal levels (in this case, current) can be analysed to determine the sequence of the polymer to be analysed. This will be discussed in more detail below.

The arrangement for the electronic circuit 4 illustrated in FIG. 5 requires a separate detection channel 40 for each sensor element 30 which is expensive to implement. Another arrangement for the electronic circuit 4 which reduces the number of detection channels 40 is illustrated in FIG. 6.

In this arrangement, the number of sensor elements 30 in the array is greater than the number of detection channels 40 and the biochemical sensing system is operable to take measurements of a polymer from sensor elements selected in a multiplexed manner, in particular an electrically multiplexed manner. This is achieved by providing a switch arrangement 42 between the sensor electrodes 23 of the sensor elements 30 and the detection channels 40. FIG. 6 shows a simplified example with four sensor cells 30 and two detection channels 40, but the number of sensor cells 30 and detection channels 40 can by greater, typically much greater. For example, for some applications, the sensor device 2 might comprise a total of 4096 sensor elements 30 and 1024 detection channels 40.

The switch arrangement 42 may be arranged as described in detail in WO 2010/122293. For example, the switch arrangement 42 may comprise plural 1-to-N multiplexers each connected to a group of N sensor elements 30 and may include appropriate hardware such as a latch to select the state of the switching.

Thus, by switching of the switch arrangement 42, the nanopore device 1 may be operated to take measurements of a polymer from sensor elements 30 selected in an electrically multiplexed manner.

The switch arrangement 42 may be controlled in the manner described in WO 2010/122293 to selectively connect the detection channels 40 to respective sensor elements 30 that have acceptable quality of performance on the basis of the amplified electrical signals that are output from the detection channels 40, but in addition the switching arrangement is controlled as described further below.

This arrangement also includes a bias control circuit 41 in respect of each sensor element 30.

Although in this example, the sensor elements 30 are selected in an electrically multiplexed manner, other types of nanopore device 1 could be configured to switch between sensor elements in a spatially multiplexed manner, for example by movement of a probe used to take electrical measurements, or by control of an optical system used to take optical measurements from the different spatial locations of different sensor elements 30.

The data processor 5 connected to the electronic circuit 4 is arranged as follows. The data processor 5 may be a computer apparatus running an appropriate program, may be implemented by a dedicated hardware device, or may be implemented by any combination thereof. The computer apparatus, where used, may be any type of computer system but is typically of conventional construction. The computer program may be written in any suitable programming language. The computer program may be stored on a computer-readable storage medium, which may be of any type, for example: a recording medium which is insertable into a drive of the computing system and which may store information magnetically, optically or opto-magnetically; a fixed recording medium of the computer system such as a hard drive; or a computer memory. The data processor 5 may comprise a card to be plugged into a computer such as a desktop or laptop. The data used by the data processor 5 may be stored in a memory 10 thereof in a conventional manner.

The data processor 5 controls the operation of the electronic circuit 3. As well as controlling the operation of the detection channels 41, the data processor controls the bias control circuits 41 and controls the switching of the switch arrangement 31. The data processor 5 also receives and processes the series of measurements from each detection channel 40. The data processor 5 stores and analyses the series of measurements, as described further below.

The data processor 5 controls the bias control circuits 41 to apply bias voltages that are sufficient to enable translocation of polymers through the pores 32 of the sensor elements 30. This operation of the biochemical sensor element 41 allows collection of series of measurements from different sensor elements 30 which may be analysed by the data processor 5, or by another data processing unit, to estimate the sequence of polymer units in a polymer, for example using techniques as described in WO 2013/041878. Data from different sensor elements 30 may be collected and combined.

There will now be described a method of controlling a nanopore device 1 shown in FIG. 7 that increases the speed of analysis by rejecting the polymer when no further analysis is needed and ensures that the nanopore is free from the ejected polymer. This method is implemented in the data processor 5. This method is performed in parallel in respect of each sensor element 30 from which a series of measurements is taken, that is every sensor element 30 in the first arrangement for the electronic circuit 4, and each sensor element 30 that is connected to a detection channel 40 by the switch arrangement 42 in the second arrangement for the electronic circuit 4.

In step C1, the nanopore device 1 is operated by controlling the bias control circuit 30 to apply a bias voltage across the pore 32 of the sensor element 30 that is sufficient to enable translocation of polymer. Based on the output signal from the detection channel 40, translocation is detected and measurements start to be taken. A series of measurements is taken over time. In some cases, the following steps operate on the series of raw measurements 11 taken by the sensor device 2, i.e. being a series of measurements of the type described above comprising successive groups of plural measurements that are dependent on the same k-mer without a priori knowledge of number of measurements in any group.

In other cases, as shown in FIG. 8, the raw measurements 11 are pre-processed using a state detection step SD to derive a series of measurements 12 that are used in the following steps instead of the raw measurements.

In such a state detection step SD, the series of raw measurements 11 is processed to identify successive groups of raw measurements and to derive a series of measurements 12 consisting of a predetermined number of measurements in respect of each identified group. Thus, a series of measurements 12 is derived in respect of each sequence of polymer units that is measured. The purpose of the state detection step SD is to reduce the series of raw measurements to a predetermined number of measurements associated with each k-mer to simplify the subsequent analysis. For example a noisy step wave signal, as shown in FIG. 4 may be reduced to states where a single measurement associated with each state may be the mean current. This state may be termed a level.

FIG. 9 shows an example of such a state detection step SD that looks for short-term increases in the derivative of the series of raw measurements 11 as follows.

In step SD-1, the series of raw measurements 11 is differentiated to derive its derivative.

In step SD-2, the derivative from step SD-1 is subjected to low-pass filtering to suppress high-frequency noise, which the differentiation in step SD-1 tends to amplify.

In step SD-3, the filtered derivative from step SD-2 is thresholded to detect transition points between the groups of measurements, and thereby identify the groups of raw measurements.

In step SD-4, a predetermined number of measurements is derived from each group of raw measurements identified in step SD-3. The measurements output from step SD-4 form the series of measurements 12.

The predetermined number of measurements may be one or more.

In the simplest approach, a single measurement is derived from each group of raw measurements, for example the mean, median, standard deviation or number, of raw measurements in each identified group.

In other approaches, a predetermined plural number of measurements of different natures are derived from each group, for example any two or more of the mean, median, standard deviation or number of raw measurements in each identified group. In that case, the a predetermined plural number of measurements of different natures are taken to be dependent on the same k-mer since they are different measures of the same group of raw measurements.

The state detection step SD may use different methods from that shown in FIG. 9. For example a common simplification of method shown in FIG. 9 is to use a sliding window analysis which compares the means of two adjacent windows of data. A threshold can then be either put directly on the difference in mean, or can be set based on the variance of the data points in the two windows (for example, by calculating Student's t-statistic). A particular advantage of these methods is that they can be applied without imposing many assumptions on the data.

Other information associated with the measured levels can be stored for use later in the analysis. Such information may include without limitation any of: the variance of the signal; asymmetry information; the confidence of the observation; the length of the group.

By way of example, FIG. 10a illustrates an experimentally determined series of raw measurements reduced by a moving window. In particular, FIG. 10a shows the series of raw measurements as the light line. Levels following state detection are shown overlaid as the dark line.

Step C2 is performed when a polymer has partially translocated through the nanopore, i.e. during the translocation. At this time, the series of measurements taken from the polymer during the partial translocation is collected for analysis, which is referred to herein as a “chunk” of measurements. Step C2 may be performed after a predetermined number of measurements have been taken so that the chunk of measurements is of predefined size, or may alternatively be after a predetermined amount of time. In the former case, the size of the chunk of measurements may be defined by parameters that are initialised at the start of a run, but are changed dynamically so that the size of the chunk of measurements changes.

In step C3, the chunk of measurements collected in step C2 is analysed. This analysis uses reference data 50. As discussed in more detail below, the reference data 50 is derived from at least one reference sequence of polymer units. The analysis performed in step C3 provides a measure of similarity between (a) the sequence of polymer units of the partially translocated polymer from which measurements have been taken and (b) the one reference sequence. Various techniques for performing this analysis are possible, some examples of which are described below.

The measure of similarity may indicate similarity with the entirety of the reference sequence, or with a portion of the reference sequence, depending on the application. The technique applied in step C3 to derive the measure of similarity may be chosen accordingly, for example being a global or a local method.

Also, the measure of similarity may indicate the similarity by various different metrics, provided that it provides in general terms a measure of how similar the sequences are. Some examples of specific measures of similarity that may be determined from the sequences in different ways are set out below.

In step C4, a decision is made responsive to the measure of similarity determined in step C3 either (a) to reject the polymer being measured, (b) that further measurements are needed to make a decision, or (c) to continue taking measurements until the end of the polymer.

If the decision made in step C4 is (a) to reject the polymer being measured, then the method proceeds to step C5 wherein the nanopore device 1 is controlled to reject the polymer, so that measurements can be taken from a further polymer.

Step C5 is performed differently as between the first and second arrangement of the electronic circuit 4, as follows.

In electronic circuit 4, in step C5 the bias control circuit 30 is controlled to apply a bias voltage across the pore 32 of the sensor element 30 that is sufficient to eject the polymer currently being translocated. This is assumed to eject the polymer and thereby makes the pore 32 available to receive a further polymer.

In order to ascertain whether an ejection was successful, a further step C7 is carried out to check that the pore 32 is free from the ejected polymer. The check in step C7 may comprise determining if the pore registers a measurement of open pore current after applying the bias voltage. This would be considered by the control circuit 30 as a successful ejection of the polymer.

After a successful ejection in step C5, the method returns to step C1 and so the bias control circuit 30 is controlled to apply a bias voltage across the pore 32 of the sensor element 30 that is sufficient to enable the capture and translocation of a further polymer through the pore 32.

An example of such a scenario is shown in the measurement signal of FIG. 10b. At about 2256.5 seconds a polymer to be analysed has been captured and has started to translocate through the nanopore, generating the signal seen after 2256.5 seconds. A decision at step C4 is made based on the measure of similarity determined in step C3 to reject the polymer being measured at about 2257.75 seconds. The signal is seen to drop to low current as step C5 is implemented, afterwards the signal reverts to open nanopore current (circa 200 pa, i.e. the signal shown before polymer was translocating through the nanopore). Thus, the sensor element is ready to capture and analyse another polymer, which occurs at around 2258 seconds. A decision at step C4 is made based on the measure of similarity determined in step C3 to reject the polymer being measured at about 2259.25 seconds. The signal is seen to drop to low current as step C5 is implemented, afterwards the signal reverts to open nanopore current and another polymer is captured by the sensor element.

Alternatively, the check C7 may comprise determining if, after applying the bias voltage, the signal returned to measurement of the strand. This would be considered as a failed ejection of the polymer. An example of such a scenario is shown in the measurement signal of FIG. 10c. As shown in FIG. 10c, a polymer to be analysed is captured by the sensor element just after 1616.5 seconds as the trace signal drops from an open pore reading (approximately 175 pa) to a series of chunk of measurements 63 of a polymer (approximately 75 pa). The step C4 of fitting the model to the series of the chunk of measurements 63 to provide the measure of similarity 65 is carried out as described above.

There may be an alternative scenario in which check C7 determines that there has been an unsuccessful ejection of a polymer. This is now described and these two examples are non-exhaustive.

In another scenario, the nanopore 32 can be determined unable to eject (i.e. that polymer has got stuck during translocation through the pore). An example trace from this scenario is shown in FIG. 10d.

In the event that the polymer has not ejected or the nanopore is determined to be blocked then in step C5, following the arrangement of the electronic circuit 4, the nanopore device 1 can be caused to cease taking measurements from the currently selected “discarded” or “blocked” sensor element 30 by controlling the switch arrangement 42 to disconnect the detection channel 40 that is currently connected to the sensor element 30 and to selectively connect that detection channel 40 to a different sensor element 30. At the same time, in step C5, the bias control circuit 30 is controlled to apply a bias voltage across the pore 32 of the sensor element 30 that is sufficient to eject the polymer currently being translocated through the currently selected sensor element 30 so that sensor element 30 is available to receive a further polymer in the future.

The method then returns to step C1 which is applied to the newly selected sensor element 30 so that the nanopore device 1 starts taking measurements therefrom.

If the decision made in step C4 is (b) that further measurements are needed to make a decision, then the method reverts to step C2. Thus, measurements of the translocating polymer continue to be taken until a chunk of measurements is next collected in step C2 and analysed in step C3. The chunk of measurements collected when step C2 is performed again may be solely the new measurements to be analysed in isolation, or may be the new measurements combined with previous chunks of measurements.

If the decision made in step C4 is (c) to continue taking measurements until the end of the polymer, then the method proceeds to step C6 without repeating the steps C2 and C3 so that no further chunks of data are analysed. The sensor element 1 continues to be operated so that measurements continue to be taken until the end of the polymer. Thereafter the method reverts to step C1, so that a further polymer may be analysed.

The degree of similarity, as indicated by the measure of similarity, which is used as the basis for the decision in step C4 may vary depending on the application and the nature of the reference sequence. Thus provided that the decision is responsive to the measure of similarity, there is in general no limitation on the degree of similarity that is used to make the different decisions.

Some examples of how the dependence on the measure of similarity might vary are as follows.

In applications where the reference sequence of polymer units is an unwanted sequence, and in step C4 a decision to reject the polymer is made to responsive to the measure of similarity indicating that the partially translocated polymer is the unwanted sequence, a relatively high degree of similarity may be used as the basis to reject the polymer. Similarly, the degree of similarity may vary depending on the nature of the reference sequence in the context of the application. Where it is intended to distinguish between similar sequences a higher degree of similarity may be required as the basis for the rejection.

Conversely, in applications where the reference sequence of polymer units from which the reference data 50 is derived is a target, and in step C4 a decision to reject the polymer is made to responsive to the measure of similarity indicating that the partially translocated polymer is not the target, a relatively low degree of similarity may be used as the basis to reject the polymer.

As another example, if the application is to determine whether a known gene from a known bacterium is present in a sample of various bacteria, the degree of similarity required to determine whether a polynucleotide has the same sequence as the target will be higher if the gene has a conserved sequence across different bacterial strains than if the sequence was not conserved.

Similarly in some of the embodiments of the invention the measure of similarity will equate to a degree of identity of a polymer to the target polymer, whereas in other embodiments the measure of similarity will equate to a probability that the polymer is the same as the target polymer.

The degree of similarity required as the basis for rejection may also be varied in dependence on the potential time saving, which is itself dependent on the application as described below. The false-positive rate that is acceptable may be dependent on the time saving. For example, where the potential time saving by rejecting an unwanted polymer is relatively high, it is acceptable to reject an increased proportion of polymers that are targets, provided that there is an overall time saving from rejection of polymers that are actually unwanted.

Reverting now to the method of FIG. 7, if at any point during the taking of measurements of a polymer it is detected that measurements are no longer being taken, indicating that the end of the polymer has been reached, then the method reverts immediately to step C1, so that a further polymer may be analysed.

The method shown in FIG. 7 may be varied, depending on the application. For example, in some variations, the decision in step C4 is never (c) to continue taking measurements until the end of the polymer, so that the method repeatedly collects and analyses chunks of measurements until the end of the polymer.

In another variation, in step C3 instead of using the reference data 50 and determining the measure of similarity, the decision in step C4 to reject the polymer may be based on other analysis of the series of measurements, in general on any analysis of the chunk of measurements.

In one possibility, step C3 may analyse whether the chunk of measurements is of insufficient quality, for example having a noise level that exceeds a threshold, having the wrong scaling, or being characteristic of a polymer that is damaged.

The decision in step C4 is made on the basis of that analysis, thereby rejecting the polymer on the basis on an internal quality control check. This still involves making a decision to reject a polymer based on a chunk of measurements, that is a series of measurements taken from the polymer during the partial translocation, and so is in contrast to that ejecting a polymer which causes a blockade, in which case the polymer is no longer translocating, so k-mer dependent measurements are not taken.

In another possibility, the method is modified as shown in FIG. 11. This method is the same as that of FIG. 7 except that step C3 is modified. In step C3, instead of using the reference data 50 derived from at least one reference sequence of polymer units and determining the measure of similarity, there is used a general model 60 that treats the measurements as observations of a series of k-mer states of different possible types and comprises: transition weightings 61, in respect of each transition between successive k-mer states in the series of k-mer states, for possible transitions between the possible types of k mer state; and emission weightings 62, in respect of each type of k-mer state that represent the chances of observing given values of measurements for that k-mer. Step C3 is modified so as to comprise deriving a measure of fit to the reference model 60. In this instance step C3 may comprise deriving a measure of fit to a model that treats the measurements as observations of a series of k-mer states of different possible types and comprises: transition weightings, in respect of each transition between successive k-mer states in the series of k-mer states, for possible transitions between the possible types of k mer state; and emission weightings, in respect of each type of k-mer state that represent the chances of observing given values of measurements for that k-mer. In this case the model may of the type described in WO2013/041878 and WO2018203084. Reference is made to WO2013/041878, WO2018203084 and WO2020109773 (all herein incorporated by reference in their entirety) for the details of the model, but a summary is given below. The measure of fit is derived, for example as the likelihood of the measurements being observed from the most likely sequence of k-mer states. Such a measure of fit indicates the quality of the measurements.

The decision in step C4 is made on the basis of that measure of fit, thereby rejecting the polymer on the basis on an internal quality control check.

Thus, the method causes a polymer to be rejected if the similarity to the reference sequence of polymer units indicates no further analysis of the polymer is needed or if the measurements taken from that polymer are of poor quality. This provides a significant time saving because a polymer may be rejected on-the-fly while it is still translocating through the pore 32. There are many applications where this is useful, some examples of which are described further below together with indications of the degree of possible timesaving.

The alternative methods of FIGS. 7 and 11 may be applied independently or in combination, in which case they may be applied simultaneously (for example with step C3 of both methods being performed in parallel, and the other steps being performed in common) or sequentially (for example performing the method of FIG. 11 prior to the method of FIG. 7).

There will now be described a method shown in FIG. 12 of controlling the biochemical analysis system 1 to sort polymers. This method is in accordance with the third aspect of the present invention. In this case, the sample chamber 24 contains a sample comprising the polymers, which may be of different types, and the wells 21 act as collection chambers for collecting the sorted polymers.

This method is implemented in the data processor 5. This method is performed in parallel in respect of plural sensor elements 30 in parallel, for example every sensor element 30 in the first arrangement for the electronic circuit 4, and each sensor element 30 that is connected to a detection channel 40 by the switch arrangement 42 in the second arrangement for the electronic circuit 4.

In step D1, the biochemical analysis system 1 is operated by controlling the bias control circuit 30 to apply a bias voltage across the pore 32 of the sensor element 30 that is sufficient to enable translocation of polymer. This causes a polymer to start translocation through the nanopore and during the translocation the following steps are performed. Based on the output signal from the detection channel 40, translocation is detected and a measurements start to be taken. A series of measurements of the polymer is taken from the sensor element 30 over time.

In some cases, the following steps operate on the series of raw measurements 11 taken by the sensor device 2, i.e. being a series of measurements of the type described above comprising successive groups of plural measurements that are dependent on the same k-mer without a priori knowledge of number of measurements in any group.

In other cases, the raw measurements 11 are pre-processed using a state detection step SD to derive a series of measurements 12 that are used in the following steps instead of the raw measurements. The state detection state SD may be performed in the same manner as in step C1 as described above with reference to FIGS. 8 and 9.

Step D2 is performed when a polymer has partially translocated through the nanopore, i.e. during the translocation. At this time, the series of measurements taken from the polymer during the partial translocation is collected for analysis, which is referred to herein as a “chunk” of measurements. Step D2 may be performed after a predetermined number of measurements have been taken so that the chunk of measurements is of predefined size, or may alternatively after a predetermined amount of time. In the former case, the size of the chunk of measurements may be defined by parameters that are initialised at the start of a run, but are changed dynamically so that the size of the chunk of measurements changes.

In step D3, the chunk of measurements collected in step D2 is analysed. This analysis uses reference data 50. As discussed in more detail below, the reference data 50 is derived from at least one reference sequence of polymer units. The analysis performed in step D3 provides a measure of similarity between (a) the sequence of polymer units of the partially translocated polymer from which measurements have been taken and (b) the one reference sequence. Various techniques for performing this analysis are possible, some examples of which are described below.

The measure of similarity may indicate similarity with the entirety of the reference sequence, or with a portion of the reference sequence, depending on the application. The technique applied in step D3 to derive the measure of similarity may be chosen accordingly, for example being a global or a local method.

Also, the measure of similarity may indicate the similarity by various different metrics, provided that it provides in general terms a measure of how similar the sequences are. Some examples of specific measures of similarity that may be determined from the sequences in different ways are set out below.

In step D4, a decision is made in dependence on the measure of similarity determined in step D3 either, (a) that further measurements are needed to make a decision, (b) to complete the translocation of the polymer into the well 21, or (c) to eject the polymer being measured back into the sample chamber 24.If the decision made in step D4 is (a) that further measurements are needed to make a decision, then the method reverts to step D2. Thus, measurements of the translocating polymer continue to be taken until a chunk of measurements is next collected in step D2 and analysed in step D3. The chunk of measurements collected when step D2 is performed again may be solely the new measurements to be analysed in isolation, or may be the new measurements combined with previous chunks of measurements.

If the decision made in step D4 is (b) to complete the translocation of the polymer into the well 21, then the method proceeds to step D6 without repeating the steps D2 and D3 so that no further no further analysis of measurements is performed.

In step D6, the translocation of the polymer into the well 21 is completed. As a result the polymer is collected in the well 21.

Step D6 may be performed by continuing to apply the same bias voltage across the pore 32 of the sensor element 30 that enables translocation of polymer.

Alternatively, in step D6, the bias voltage may be changed to perform the remainder of the translocation of the polymer at an increased rate to reduce the time taken for translocation. This is advantageous because it increases the overall speed of the sorting process. It is acceptable to increase the translocation speed, because the polymer no longer needs to be analysed. Typically, the change in bias voltage may be an increase. In a typical system, the increase may be significant. For example in one embodiment, the translocation speed may be increased from around 30 bases per second to around 10,000 bases per second. The possibility of changing the translocation speed may depend on the configuration of the sensor element. For example, where a polymer binding moiety, for example an enzyme, is used to control the translocation, this may depend on the a polymer binding moiety used. Advantageously, a polymer binding moiety that can control the rate may be selected.

During step D6, the sensor element 1 may continue to be operated so that measurements continue to be taken until the end of the polymer, but this is optional as there is no need to determine the remainder of the sequence.

After step D6, the method reverts to step D1, so that a further polymer may be translocated.

If the decision made in step D4 is (c) to eject the polymer, then the method proceeds to step D5 wherein the biochemical analysis system 1 is controlled to eject the polymer being measured back into the sample chamber 24, so that measurements can be taken from a further polymer.

In step D5, the bias control circuit 30 is controlled to apply a bias voltage across the pore 32 of the sensor element 30 that is sufficient to eject the polymer currently being translocated. This ejects the polymer and thereby makes the pore 32 available to receive a further polymer. After ejection such ejection in step D5, the method returns to step D1 and so the bias control circuit 30 is controlled to apply a bias voltage across the pore 32 of the sensor element 30 that is sufficient to enable translocation of a further polymer through the pore 32.

On reverting to step D1, the method repeats. Repeated performance of the method causes successive polymers from the sample chamber 24 to be translocated and processed.

Thus, the method makes use of the measure of similarity provided by the analysis of the series of measurements taken from the polymer during the partial translocation as the basis for whether or notusccessive polymers are collected in the well 21. In this manner, polymers from the sample in the sample chamber 24 are sorted and desired polymers are selectively collected in the well 21.

The collected polymers may be recovered. This may be done after the method has been run repeatedly, by removing the sample from the sample chamber 24 and then recovering the polymers from the wells 21. Alternatively, this could be done during translocation of polymers from the sample, for example by providing the biochemical analysis system 1 with a fluidics system that extracts the polymers from the wells 21.

The method may be applied to a wide range of applications. For example, the method could be applied to polymers that are polynucleotides, for example viral genomes or plasmids. A viral genome typically has a length of order 10-15 kB (kilobases) and a plasmid typically has a length of order 4 kB. In such examples, the polynucleotides would not have to be fragmented and could be collected whole. The collected viral genome or plasmid could be used in any way, for example to transfect a cell. Transfection is the process of introducing DNA into a cell nucleus and is an important tool used in studies investigating gene function and the modulation of gene expression, thus contributing to the advancement of basic cellular research, drug discovery, and target validation. RNA and proteins may also be transfected.

The degree of similarity, as indicated by the measure of similarity, that is used as the basis for the decision in step D4 may vary depending on the application and the nature of the reference sequence. Thus provided that the decision is dependant on the measure of similarity, there is in general no limitation on the degree of similarity that is used to make the different decisions.

Some examples of how the dependence on the measure of similarity might vary are as follows.

In many applications, the reference sequence of polymer units from which the reference data 50 is derived is a wanted sequence. In that case, in step D4 a decision to complete the translocation is made to responsive to the measure of similarity indicating that the partially translocated polymer is the wanted sequence, a relatively high degree of similarity may be used as the basis to complete the translocation.

However, this is not essential. In some applications, the reference sequence of polymer units is an unwanted sequence. In that case, in step D4 a decision to complete the translocation is made to responsive to the measure of similarity indicating that the partially translocated polymer is not the unwanted sequence.

Similarly, the degree of similarity may vary depending on the nature of the reference sequence in the context of the application. Where it is intended to distinguish between similar sequences a higher degree of similarity may be required as the basis for the rejection.

The method may be performed using the same reference data 50 and the same criteria in step D4 in respect of each sensor element 30. In that case, each well 21 collects the same polymers in parallel.

Alternatively, the method may be performed to collect different polymers in different wells 21. In this case, differential sorting is performed. In one example of this, different reference data 50 is used in respect of different sensor elements 30. In another example of this, the same reference data 50 is used in respect of different sensor elements 30, but step D4 is performed with different dependence on the measure of similarity in respect of different sensor elements.

The methods shown in FIGS. 7, 11 and 12 may be varied, depending on the application.

A variety of different types of reference sequence of polymer units may be used, depending on the application. Without limitation, where the polymer is a polynucleotide, the reference sequence of polymer units may comprise one or more reference genomes or a region of interest of the one or more genomes to which the measurement is compared.

The source of the reference data 50 may vary depending on the application. The reference data may be generated from the reference sequence of polymer units or from measurements taken from the reference sequence of polymer units.

In some applications, the reference data 50 may be pre-stored having been generated previously. In other applications, the reference data 50 is generated at the time the method is performed.

The reference data 50 may be provided in respect of a single reference sequence of polymer units or plural reference sequences of polymer units. In the latter case, either step C3 is performed in respect of each sequence or else one of the plural reference sequences is selected for use in step C3. In the latter case, the selection may be made based on various criteria, depending on the application. For example, the reference data 50 may be applicable to different types of nanopore device 1 (e.g. different nanopores) and/or ambient conditions, in which case the selection of the reference model 8 is based on the type of nanopore device 1 actually used and/or the actual ambient conditions.

The nanopore device 1 described above is an example of a nanopore device that comprises an array of sensor elements that each comprise a nanopore. However, the method may be generalised to any nanopore device that is operable to take successive measurements of polymers selected in a multiplexed manner, without the use of nanopores. An example of such nanopore device is a scanning probe microscope, which may be an atomic force microscope (AFM), a scanning tunnelling microscope (STM) or another form of scanning microscope. In such a case, the nanopore device may be operable to take successive measurements of polymers selected in a spatially multiplexed manner. For example, the polymers may be disposed on a substrate in different spatial locations and the spatial multiplexing may be provided by movement of the probe of the scanning probe microscope.

In the case where the reader is an AFM, the resolution of the AFM tip may be less fine than the dimensions of an individual polymer unit. As such the measurement may be a function of multiple polymer units. The AFM tip may be functionalised to interact with the polymer units in an alternative manner to if it were not functionalised. The AFM may be operated in contact mode, non-contact mode, tapping mode or any other mode.

In the case where the reader is a STM the resolution of the measurement may be less fine than the dimensions of an individual polymer unit such that the measurement is a function of multiple polymer units. The STM may be operated conventionally or to make a spectroscopic measurement (STS) or in any other mode.

The reference data 50 may take various forms that are derived from the reference sequence of polymer units in different ways. The analysis performed in step C4 to provide the measure of similarity is dependent on the form of the reference data 50. Some non-limitative examples will now be described.

In a first example, the reference data 50 represents the identity of the polymer units of the at least one reference sequence. In that case, step C4 comprises the process shown in FIG. 13, as follows.

In step C4a-1, the chunk of measurements 63 is analysed to provide an estimate 64 of the identity of the polymer units of a sequence of polymer units of the partially translocated polymer. Step C4a-1 may in general be performed using any method for analysing the measurements taken by the nanopore device.

Step C4a-1 may be performed in particular using the method described in detail in

WO-2013/041878, WO2018203084 and WO2020109773. Reference is made to WO 2013/041878 for the details of the method, but a summary is given as follows.

This method makes reference to a general model 60 comprises transition weightings 61 and emission weightings 62 in respect of a series of k-mer states corresponding to the chunk of measurements 63.

The transition weightings 61 are provided in respect of each transition between successive k-mer states in the series of k-mer states. Each transition may be considered to be from an origin k-mer state to a destination k-mer state. The transition weightings 61 represent the relative weightings of possible transitions between the possible types of the k-mer state, which is from an origin k-mer state of any type to a destination k-mer state of any type. In general, this includes a weighting for a transition between two k-mer states of the same type.

The emission weightings 62 are provided in respect of each type of k-mer state. The emission weightings 62 are weightings for different measurements being observed when the k-mer state is of that type. Conceptually, the emission weightings 62 may be thought of as representing the chances of the chances of observing given values of measurements for that k-mer state, although they do not need to be probabilities.

Conceptually, the transition weightings 61 may be thought of as representing the chances of the possible transition, although they do not need to be probabilities. Therefore, the transition weightings 61 take account of the chance of the k-mer state on which the measurements depend transitioning between different k-mer states, which may be more or less likely depending on the types of the origin and destination k-mer states.

By way of example and without limitation, the model may be an HMM in which the transition weightings 61 and emission weightings 62 are probabilities.

Step C4a-1 uses the reference model 60 to derive an estimate 64 of the identity of the polymer units of a sequence of polymer units of the partially translocated polymer. This may be performed using known techniques that are applicable to the nature of the reference model 60. Typically, such techniques derive the estimate 64 based on the likelihood of the measurements predicted by the reference model 50 being observed from sequences of k-mer states.

Such methods may also provide a measure of fit of the measurements to the model, for example a quality score that indicates the likelihood of the measurements predicted by the reference model 50 being observed from the most likely sequence of k-mer states. Such measures are typically derived because they are used to derive the estimate 64.

As an example in the case that the general model 60 is an HMM, the analytical technique may be a known algorithm for solving the HMM, for example the Viterbi algorithm which is well known in the art. In that case, the estimate 64 is derived based on the likelihood predicted by the general model 60 being produced by overall sequences of k-mer states.

As another example in the in the case that the general model 60 is an HMM, the analytical technique may be of the type disclosed in Fariselli et al., “The posterior-Viterbi: a new decoding algorithm for hidden Markov models”, Department of Biology, University of Casadio, archived in Cornell University, submitted 4 Jan. 2005. In this method, a posterior matrix (representing the probabilities that the measurements are observed from each k-mer state) and obtain a consistent path, being a path where neighbouring k-mer states are biased towards overlapping, rather than simply choosing the most likely k-mer state per event. In essence, this allows recovery of the same information as obtained directly from application of the Viterbi algorithm.

The above description is given in terms of a general model 60 that is an HMM in which the transition weightings 61 and emission weightings 62 are probabilities and method uses a probabilistic technique that refers to the general model 60. However, it is alternatively possible for the general model 60 to use a framework in which the transition weightings 61 and/or the emission weightings 62 are not probabilities but represent the chances of transitions or measurements in some other way. In this case, the method may use an analytical technique other than a probabilistic technique that is based on the likelihood predicted by the general model 60 of the series of measurements being produced by sequences of polymer units. The analytical technique may explicitly use a likelihood function, but in general this is not essential.

In step C4a-2, the estimate 64 is compared with the reference data 50 to provide the measure of similarity 65. This comparison may use any known technique for comparing two sequence of polymer units, typically being an alignment algorithm that derives an alignment mapping between the sequence of polymer units, together with a score for the accuracy of the alignment mapping which is therefore the measure of similarity 65. Any of a number of available fast alignment algorithms may be used, such as Smith-Waterman alignment algorithm, BLAST or derivatives thereof, or a k-mer counting technique.

This example of the form of the reference data 50 has the advantage that the process for deriving the measure of similarity 65 is rapid, but other forms of the reference data are possible.

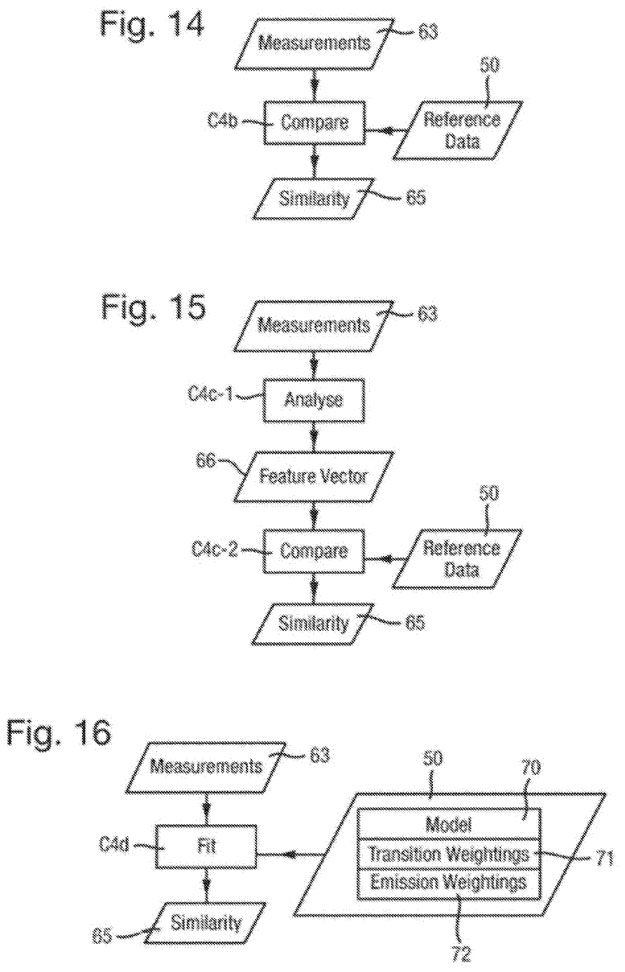

In a second example, the reference data 50 represents actual or simulated measurements taken by the nanopore device 1. In that case, step C4 comprises the process shown in FIG. 14 which simply comprises step C4b of comparing the chunk of measurements 63 with the reference data 50 to derive the measure of similarity 65. Any suitable comparison may be made, for example using a distance function to provide a measure of the distance between the two series of measurements, as the measure of similarity 65.

In a third example, the reference data 50 represents a feature vector of time-ordered features representing characteristics of the measurements taken by the nanopore device 1. Such a feature vector may be derived as described in detail in WO-2013/121224 to which reference is made and which is incorporated herein by reference. In that case, step C4 comprises the process shown in FIG. 15 which is performed as follows.

In step C4c-1, the chunk of measurements 63 is analysed to derive a feature vector 66 of time-ordered features representing characteristics of the measurements.

In step C4c-2, the feature vector 66 is compared with the reference data 50 to derive the measure of similarity 65. The comparison may be performed using the methods described in detail in WO-2013/121224.

In a fourth example, the reference data 50 represents a reference model 70. In that case, step C4 comprises the process shown in FIG. 16 which comprises step C4d of fitting the reference model 70 to the series of the chunk of measurements 63 to provide the measure of similarity 65 as the fit of the reference model 70 to the chunk of measurements 63. This may be performed as follows.

The reference model 70 is a model of the reference sequence of polymer units in the nanopore device 1. The reference model 70 treats the measurements as observations of a reference series of k-mer states corresponding to the reference sequence of polymer units. The k-mer states of the reference model 70 may model the actual k-mers on which the measurements depend, although mathematically this is not necessary and so the k-mer states may be an abstraction of the actual k-mers. Thus, the different types of k-mer states may correspond to the different types of k-mers that exist in the reference sequence of polymer units.

The reference model 70 may be considered as an adaption of the general model 60 to model the measurements that are obtained specifically when the reference sequence is measured. Thus, reference model 70 treats the measurements as observations of a reference series of k-mer states 73 corresponding to the reference sequence of polymer units. As such, the reference model 70 has the same form as the general model 60, in particular comprising transition weightings 71 and emission weightings 72 as will now be described.

The transition weightings 71 represent transitions between the k-mer states 73 of the reference series. Those k-mer states 73 correspond to the reference sequence of polymer units. Thus, successive k-mer states 73 in the reference series corresponds to a successive overlapping groups of k polymer units. As such there is an intrinsic mapping between the k-mer states 73 of the reference series and the polymer units of the reference sequence. Similarly, each k-mer states 73 is of a type corresponding to the combination of the different types of each polymer unit in the group of k polymer units.

This is illustrated with reference to the state diagram of FIG. 17 which shows an example of three successive k-mer states 73 in the reference series of estimated k-mer states 73. In this example, k is three and the reference sequence of polymer units includes successive polymer units labelled A, A, C, G, T. (although of course those specific types of the k-mer states 73 are not limitative). Accordingly, the successive k-mer states 73 of the reference series corresponding to those polymer units are of types AAC, ACG, CGT which correspond to a measured sequence of polymer units AACGT.

The state diagram of FIG. 18 illustrates transitions between the k-mer states 73 of the reference series, as represented by the transition weightings 71. In this example, states may only forwards progress through the k-mer states 73 of the reference series is allowed (although in general backwards progression could additionally be allowed). Three different types of transition 74, 75 and 76 are illustrated as follows.

From each given k-mer state 73 in the reference series, a transition 74 to the next k-mer state 73 is allowed. This models the likelihood of successive measurements in the series of measurements 12 being taken from successive k-mers of the reference sequence of polymer units. In the case that the chunk of measurements 63 are pre-processed to identify successive groups of measurements and to derive a series of processed measurements for further analysis, consisting of a predetermined number of measurements in respect of each identified group, the transition weightings 71 represent this transition 74 as having a relatively high likelihood.

From each given k-mer state 73 in the reference series, a transition 75 to the same k-mer state is allowed. This models the likelihood of successive measurements in the series of measurements 12 being taken from the same k-mers of the reference sequence of polymer units. This may be referred to as a “stay”. In the case that the chunk of measurements 63 are pre-processed to identify successive groups of measurements and to derive a series of processed measurements, consisting of a predetermined number of measurements in respect of each identified group, the transition weightings 71 represent this transition 75 as having a relatively low likelihood compared to the transition 74.

From each given k-mer state 73 in the reference series, a transition 76 to the subsequent k-mer states 73 beyond the next k-mer state 73 is allowed. This models the likelihood of no measurement being taken from the next k-mer state, so that successive measurements in the series of measurements 12 being taken from k-mers of the reference sequence of polymer units that are separated. This may be referred to as a “skip”. In the case that the chunk of measurements 63 are pre-processed to identify successive groups of measurements and to derive a series of processed measurements, consisting of a predetermined number of measurements in respect of each identified group, the transition weightings 71 represent this transition 76 as having a relatively low likelihood compared to the transition 74.

The level of the transition weightings 71 representing the transitions 75 and 76 for skips and stays relative to the level of the transition weightings 71 representing the transitions 74 may be derived in the same manner as the transition weightings 61 for skips and stays in the general model 31, as described above.

In the alternative that the chunk of measurements 63 are not pre-processed to identify successive groups of measurements and to derive a series of processed measurements, so that the further analysis is performed on the chunk of measurements 63 themselves, then the transition weightings 71 are similar but are adapted to increase the likelihood of the transition 75 representing a skip to represent the likelihood of successive measurements being taken from the same k-mer. The level of the transition weightings 71 for the transition 75 are dependent on the number of measurements expected to be taken from any given k-mer and may be determined by experiment for the particular nanopore device 1 that is used.

Emission weightings 72 are provided in respect of each k-mer state. The emission weightings 72 are weightings for different measurements being observed when the k-mer state is observed. The emission weightings 72 are therefore dependent on the type of the k-mer state in question. In particular, the emission weightings 72 for a k-mer state of any given type are the same as the emission weightings 62 for that type of k-mer state in the general model 60 as described above.

Step C4d of fitting the model to the series of the chunk of measurements 63 to provide the measure of similarity 65 as the fit of the reference model 70 to the chunk of measurements 63 is performed using the same techniques as described above with reference to FIG. 7, except that the reference model 70 replaces the general model 60.

As a result of the form of the reference model 70, in particular the representation of transitions between the reference series of k-mer states 73, the application of the model intrinsically derives an estimate of an alignment mapping between the chunk of measurements 63 and the reference series of k-mer states 73. This may be understood as follows. As the general model 60 represents transitions between the possible types of k-mer state, the application of the model provides estimates of the type of k-mer state from which each measurement is observed. As the reference model 70 represents transitions between the reference series of k-mer states 73, the application of the reference model 70 instead estimates the k-mer state 73 of the reference sequence from which each measurement is observed, which is an alignment mapping between the series of measurements and the reference series of k-mer states 73.

In addition, the algorithm derives a score for the accuracy of the alignment mapping, for example representing the likelihood that the estimate of the alignment mapping is correct, for example because the algorithm derives the alignment mapping based on such a score for different paths through the model. Thus, this score for the accuracy of the alignment mapping is therefore the measure of similarity 65

As an example in the case that the reference model 70 is an HMM and the analytical technique applied is the Viterbi algorithm as described above, then the score is simply the likelihood predicted by the reference model 70 associated with the derived estimate of the alignment mapping.

As another example in the in the case that the general model 60 is an HMM, the analytical technique may be of the type disclosed in Fariselli et al., “The posterior-Viterbi: a new decoding algorithm for hidden Markov models”, Department of Biology, University of Casadio, archived in Cornell University, submitted 4 Jan. 2005, as described above. This again derives a score that is the measure of similarity 65.

The reference model 70 may be generated from the reference sequence of polymer units or from measurements taken from the reference sequence of polymer units, as follows.

The reference model 70 may be generated from a reference sequence of polymer units 80 by the process shown in FIG. 19, as follows. This is useful in applications where the reference sequence is known, for example from a library or from earlier experiments. The input data representing the reference sequence of polymer units 80 may already be stored in the data processor 5 or may be input thereto.

This process uses stored emission weightings 81 which comprise the emission weightings e1 to en in respect of a set of possible types of k-mer state type-1 to type-n. Advantageously, this allows generation of the reference model for any reference sequence of polymer units 80, based solely on the stored emission weightings 81 for the possible types of k-mer state.

The process is performed as follows.

In step P1, the reference sequence of polymer units 80 is received and a reference sequence of k-mer states 73 is generated therefrom. This is a straightforward process of establishing, for each k-mer state 73 in the reference sequence, the type of that k-mer state 73 based on the combination of types of polymer unit 80 to which that k-mer state 73 corresponds.

In step P2, the reference model is generated, as follows.

The transition weightings 71 are derived for transitions between the reference series of k-mer states 73 derived in step P1. The transition weightings 71 take the form described above, defined with respect to the reference series of k-mer states 73.

The emission weightings 72 are derived for each k-mer state 73 in the series of k-mer states 73 derived in step P1, by selecting the stored emission weightings 81 according to the type of the k-mer state 73. For example, if a given k-mer state 73 is of type type-4, then the emission weightings e4 are selected.

The reference model 70 may be generated from a series of reference measurements 93 taken from the reference sequence of polymer units by the process shown in FIG. 20, as follows. This is useful, for example, in applications where the reference sequence of polymer units is measured contemporaneously with the target polymer. In particular, in this example there is no requirement that the identity of the polymer units in the reference sequence are themselves known. The series of reference measurements 93 may be taken from the polymer that comprises the reference sequence of polymer units by the nanopore device 1.

This process uses a further model 90 that treats the series of reference measurements as observations of a further series of k-mer states of different possible types. This further model 90 is a model of the nanopore device 1 used to take the series of reference measurements 93 and may be identical to the general model 60 described above, for example of the type disclosed in WO-2013/041878. Thus, the further model comprises transition weightings 91 in respect of each transition between successive k-mer states in the further series of k-mer states, that are transition weightings 91 for possible transitions between the possible types of the k-mer states; and emission weightings 92 in respect of each type of k-mer state, being emission weightings 92 for different measurements being observed when the k-mer state is of that type.

The process is performed as follows.

In step Q1, the further model 90 is applied to the series of reference measurements 93 to estimate the reference series of k-mer states 73 as a series of discrete estimated k-mer states. This may be done using the techniques described above.

In step Q2, the reference model 70 is generated, as follows.

The transition weightings 71 are derived for transitions between the reference series of k-mer states 73 derived in step Q1. The transition weightings 71 take the form described above, defined with respect to the reference series of k-mer states 73.

The emission weightings 72 are derived for each k-mer state 73 in the series of k-mer states 73 derived in step Q1, by selecting the emission weightings from the weightings of the further model 50 according to the type of the k-mer state 73. Thus, the emission weightings for each type of k-mer state 73 in the reference model are the same as the emission weightings for that type of k-mer state 73 in the further model 50.

Examples of various applications have been explored from the disclosure of WO2016059427 which is herein incorporated by reference in its entirety for the basis of the decision in step C4 and an indication of possible time-savings. The polymers are polynucleotides and the usual assumption has been made that measurement of the first 250 nucleotides followed by comparison to a reference sequence will be enough to determine a) whether it relates to that reference sequence or not and b) its location with respect to the overall sequence. However it may be more or less than this number. The number of polymer units required to make a determination will not necessarily be fixed. Typically measurements will be continually carried out on a continual basis until such a determination can be made.

For each of the types of application, there might be a slightly different use of the method shown in FIG. 7. A mixture of the types of application might also be used. The analysis performed in step C3 and/or the basis of the decision in step C4 might also be adjusted dynamically as the run proceeds. For example, there might be no decision logic applied initially, then logic is used later into the run when enough data has built up to make decisions. Alternatively, the decision logic may change during a run.

The method shown in FIG. 16 results in generation of an alignment mapping. This method may be applied more generally as follows.

FIG. 21 shows a method of estimating an alignment mapping between (a) a series of measurements of a polymer comprising polymer units, and (b) a reference sequence of polymer units. The method is performed as follows.

As shown in FIG. 21, the input to the method may be a series of measurements 12 derived by taking a series of raw measurements from a sequence of polymer units by the biochemical analysis system 1 and subjecting them to pre-processing as described above. As an alternative, the input to the method may be a series of raw measurements 11.

The method uses the reference model 70 of the reference sequence of polymer units, the reference model 70 being stored in the memory 10 of the data processor 5. The reference model 70 takes the same form as described above, treating the measurements as observations of a reference series of k-mer states corresponding to the reference sequence of polymer units.

The reference model 70 is used in alignment step S1. In particular, in alignment step S1, the reference model 70 is applied to the series of measurements 12. Alignment step S1 is performed in the same manner as step C4d above. In other words, alignment step S1 is performed by fitting the model to the series of the chunk of measurements 63 to provide the measure of similarity 65 as the fit of the reference model 70 to the chunk of measurements 63 is performed using the same techniques as described above with reference to FIG. 13, except that the reference model 70 replaces the general model 60.

As a result of the form of the reference model 70, in particular the representation of transitions between the reference series of k-mer states 73, the application of the model intrinsically derives an estimate 13 of an alignment mapping between the series of measurements and the reference series of k-mer states 73. This may be understood as follows. As the general model 60 represents transitions between the possible types of k-mer state, the application of the model provides estimates of the type of k-mer state from which each measurement is observed, i.e. the initial series of estimates of k-mer states 34 and the discrete estimated k-mer states 35 which each estimate the type of the k-mer state from which each measurement is observed. As the reference model 70 represents transitions between the reference series of k-mer states 73, the application of the reference model 70 instead estimates the k-mer state 73 of the reference sequence from which each measurement is observed, which is an alignment mapping between the series of measurements and the reference series of k-mer states 73.

As there is an intrinsic mapping between the k-mer states 73 of the reference series and the polymer units of the reference sequence, the alignment mapping between the series of measurements and the reference series of k-mer states 73 also provides an alignment mapping between the series of measurements and the reference sequence of polymer units.

FIG. 22 illustrates an example of an alignment mapping to illustrate its nature. In particular, FIG. 22 shows an alignment mapping between polymer units p0 to p7 of the reference sequence, k-mer states k1 to k6 of the reference series and measurements m1 to m7. By way of illustration in this example k is three. The horizontal lines indicate an alignment between a k-mer state and a measurement, or in the case of a dash an alignment to a gap in the other series. Thus, inherently the polymer units p0 to p7 of the reference sequence are aligned to k-mer states k1 to k6 of the reference series as illustrated. K-mer state k1 corresponds to, and is mapped to, polymer units p1 to p3 and so on. As to the mapping between k-mer states k1 to k6 of the reference series and measurements m1 to m7: k-mer state k1 is mapped to measurement m1, k-mer state k2 is mapped to measurement m2, k-mer state k3 is mapped to a gap in the series of measurements, k-mer state k4 is mapped to measurement m3, and measurements m4 and m5 are mapped to a gap in the series of k-mer states.

Depending on the method applied, the form of the estimate 13 of the alignment mapping may vary which has been explored from the disclosure of WO2016059427, which is herein incorporated by reference in its entirety.

In all applications the improved method of the present invention allows for a more efficient and effective analysis of desired polymers. This is particularly pertinent for the analysis of longer polymers to prevent nanopore device resource being used on analysis of polymers that are not of interest.

1. A method of controlling a nanopore device for analysing a polymer, the nanopore device comprising at least one sensor element, the sensor element comprising a nanopore and a sensor, the method comprising:
translocating a polymer through the nanopore;
generating a series of measurements using the sensor as the polymer translocates through the nanopore;
comparing the series of measurements to a reference data to determine a measurement of similarity;
operating the nanopore device to eject the polymer from the nanopore if the measure of similarity is determined to be below a threshold value; and
determining whether the polymer has been successfully ejected from the nanopore by analysing a further set of measurements taken by the sensor;
wherein if it has been determined that the polymer has not been successfully ejected from the nanopore, the method further comprises operating the nanopore device to perform either:
(i) At least one additional step to eject the polymer from the nanopore; or
(ii) ceasing the taking of measurements from the nanopore.

2. The method according to claim 1, wherein the further series of measurements used to determine whether the polymer has been successfully ejected from the nanopore are compared to a predetermined value.

3. The method according to claim 2, wherein the predetermined value is a measurement by the sensor when the nanopore is free from polymer.

4. The method according to any one of the previous claims, wherein the at least one sensor element is operable to eject a polymer that is translocating through the nanopore.

5. A method according to claim 4, wherein the sensor comprises an electrode, and the at least one sensor element is operable to eject a polymer that is translocating through the nanopore by the at least one additional step, the at least one additional step comprising the application of an ejection bias voltage to eject the polymer, such that the step of operating the sensor element to eject the polymer from the nanopore is performed by applying an ejection bias voltage.

6. The method according to claim 5, wherein the method comprises an additional step if the polymer has been successfully ejected: operating the sensor element to accept a further polymer to translocate through the nanopore, wherein operating the sensor element to accept a further polymer is performed by applying a translocation bias voltage sufficient to enable translocation of a further polymer therethrough.

7. The method according to claim 5, comprising an additional step if the polymer has not been successfully ejected: the application of a second ejection bias voltage to eject the polymer, the second ejection bias voltage being higher than the first ejection bias voltage.

8. The method according to claim 6, comprising an additional step if the polymer has not been successfully ejected after application of the second ejection bias voltage: the application of a third ejection bias voltage to eject the polymer, wherein the third ejection bias voltage is higher than the second ejection bias voltage.

9. A method according to any one of claims 5 to 8, wherein if the polymer has not been successfully ejected then the method further comprises, when operating the nanopore device to cease taking measurements from the sensor element.

10. The method of according to any previous claim, wherein the nanopore device comprises an alternative sensor element, and following operating the nanopore device to cease taking measurements from the sensor element, the method further comprising selecting the alternative sensor element and operating the device to start taking measurements from the alternative sensor element.

11. The method of according to any previous claim, wherein the nanopore device comprises: a detection circuit comprising an array of detection channels each capable of taking electrical measurements from an array sensor element, the number of sensor elements in the array being greater than the number of detection channels; and a switch arrangement capable of selectively connecting the detection channels to respective sensor elements in a multiplexed manner; wherein when the method further comprises operating the nanopore device to perform ceasing the taking of measurements from the nanopore, then the method further comprises switching via the switching arrangement to an alternative detection channel in the array of detection channels.

12. A method according to any one of the preceding claims, wherein the reference data derived from at least one reference sequence of polymer units represents actual or simulated measurements taken by a nanopore device, and said step of analysing the series of measurements taken from the polymer during the partial translocation comprises: comparing the series of measurements with the reference data.

13. A method according to any one of claims 1 to 12, wherein the reference data derived from at least one reference sequence of polymer units represents a feature vector of time-ordered features representing characteristics of the measurements taken by a biochemical analysis system, and said step of analysing the series of measurements taken from the polymer during the partial translocation comprises: deriving, from the series of measurements, a feature vector of time-ordered features representing characteristics of the measurements, and comparing the derived feature vector with the reference data.

14. A method according to any one of claims 1 to 12, wherein the reference data derived from at least one reference sequence of polymer units represents the identity of the polymer units of the at least one reference sequence, and said step of analysing the series of measurements taken from the polymer during the partial translocation comprises: analysing the series of measurements to provide an estimate of the identity of the polymer units of a sequence of polymer units of the partially translocated polymer, and comparing the estimate with the reference data to provide the measure of similarity.

15. A method according to any one of claims 1 to 12, wherein the measurements are dependent on a k-mer, being k polymer units of polymer, where k is an integer; the reference data represents a reference model that treats the measurements as observations of a reference series of k-mer states corresponding to the reference sequence of polymer units, wherein the reference model comprises: transition weightings for transitions between the k-mer states in the reference series of k-mer states; and in respect of each k-mer state, emission weightings for different measurements being observed when the k-mer state is observed, and said step of analysing the series of measurements taken from the polymer during the partial translocation comprises fitting the model to the series of the series of measurements to provide the measure of similarity as the fit of the model to the series of measurements.

16. A method according to claim 15, wherein the measurements are dependent on a k-mer, being k polymer units of polymer, where k is an integer.

17. A method according to any one of the preceding claims, wherein the nanopore is a biological pore.

18. A method according to any one of the preceding claims, wherein the polymer comprises a series of polymer units to be identified by the nanopore device.

19. A method according claim 18, wherein the polymer is a polynucleotide, and the polymer units are nucleotides.

20. A method according to any one of the preceding claims, wherein the translocation of said polymer through a nanopore is performed in a ratcheted manner.

21. A method according to any one of the preceding claims, wherein the nanopore device comprises a sensor electrode and the measurements comprise electrical measurements.

22. The method according to any one of the previous claims, wherein the nanopore device comprises a sensor electrode, and the measurements taken by the sensor are indicative of ion flow through the nanopore.

23. A nanopore device for analysing polymers that comprise a sequence of polymer units, wherein the nanopore device comprises at least one sensor element that comprises a nanopore, a sensor and a data processor, and the nanopore device is operable to take successive measurements of a polymer from a sensor element, during translocation of the polymer through the nanopore of the sensor element, wherein the data processor of the nanopore device is arranged, when a polymer has partially translocated through the nanopore, to analyse the series of measurements taken from the polymer during the partial translocation thereof, comparing the series of measurements to a reference data to determine a measurement of similarity; wherein the data processor of the nanopore device is further arranged, responsive to the measure of similarity, to eject the polymer and determine whether the polymer has been successfully ejected from the nanopore by analysing a further set of measurements taken by the sensor.

24. A nanopore device according to claim 23, wherein the data processor is connected to the sensor element via a control circuit.

25. A nanopore device according to claims 23 or 24, wherein the nanopore is a biological pore.

26. A nanopore device according to any one claims 23 to 25, wherein the polymer comprises a series of polymer units to be identified by the nanopore device.

27. A nanopore device according to claim 26, wherein the polymer is a polynucleotide, and the polymer units are nucleotides.

28. A nanopore device according to any one claims 23 to 27, wherein the translocation of said polymer through a nanopore is performed in a ratcheted manner.

29. A nanopore device according to any one claims 23 to 28, wherein the nanopore device comprises a sensor electrode and the measurements comprise electrical measurements.

30. A nanopore device according to any one claims 23 to 29, wherein the nanopore device comprises a sensor electrode, and the measurements taken by the sensor are indicative of ion flow through the nanopore.

31. A nanopore device according to any one of claims 23 to 30, wherein the nanopore device comprises: a detection circuit comprising an array of detection channels each capable of taking electrical measurements from an array of sensor elements, the number of sensor elements in the array being greater than the number of detection channels; and a switch arrangement capable of selectively connecting the detection channels to respective sensor elements in a multiplexed manner.

32. A nanopore device according to any one of claims 23 to 32, wherein if it has been determined that the polymer has not been successfully ejected from the nanopore, the data processor is further arranged to operate the nanopore device to perform either:
(iii) At least one additional step to eject the polymer from the nanopore; or
(iv) ceasing the taking of measurements from the nanopore.

33. A method of controlling a nanopore device for analysing polymers that comprise a sequence of polymer units, wherein the nanopore device comprises at least one sensor element that comprises a nanopore and a sensor, and the nanopore device is operable to take successive measurements of a polymer from a sensor element, during translocation of the polymer through the nanopore of the sensor element, and the nanopore device is operable to generate successive measurements of a polymer from a sensor element, during translocation of the polymer through the nanopore of the sensor element, wherein the method comprises, when a polymer has partially translocated through the nanopore, analysing the series of measurements taken from the polymer during the partial translocation thereof by deriving a measure of fit to a model that treats the measurements as observations of a series of k-mer states of different possible types and comprises: transition weightings, in respect of each transition between successive k-mer states in the series of k-mer states, for possible transitions between the possible types of k-mer state; and emission weightings, in respect of each type of k-mer state that represent the chances of observing given values of measurements for that k-mer, and responsive to the measure of fit, operating the biochemical analysis system to eject the polymer and determine whether the polymer has been successfully ejected from the nanopore by analysing a further set of measurements taken by the sensor.

34. A nanopore device for analysing polymers that comprise a sequence of polymer units, wherein the nanopore device comprises at least one sensor element that comprises a nanopore and a sensor, and the nanopore device is operable to take successive measurements of a polymer from a sensor element, during translocation of the polymer through the nanopore of the sensor element, wherein the biochemical analysis system is arranged, when a polymer has partially translocated through the nanopore, analysing the series of measurements taken from the polymer during the partial translocation thereof by deriving a measure of fit to a model that treats the measurements as observations of a series of k-mer states of different possible types and comprises: transition weightings, in respect of each transition between successive k-mer states in the series of k-mer states, for possible transitions between the possible types of k-mer state; and emission weightings, in respect of each type of k-mer state that represent the chances of observing given values of measurements for that k-mer, and the biochemical analysis system is arranged, responsive to the measure of fit, to eject the polymer and determine whether the polymer has been successfully ejected from the nanopore by analysing a further set of measurements taken by the sensor.
